(19) United States
(12) Patent Application Publication
(10) Pub. No.: US 2025/0059704 A1
(43) Pub. Date: Feb. 20, 2025

(54) USE OF A STRUCTURAL POLYPEPTIDE FOR TREATING OR FINISHING TEXTILES

(71) Applicant: Amsilk GmbH, Neuried (DE)

(72) Inventors: Lin Romer, Ottobrunn (DE); Andreas Schmideder, Velden (DE); Joseph Lauer, Munich (DE)

(21) Appl. No.: 18/713,971

(22) PCT Filed: Dec. 6, 2022
(86) PCT No.: PCT/EP2022/084490

§ 371 Date: May 28, 2024

(30) Foreign Application Priority Data
Dec. 7, 2021 (EP) 212129944
Dec. 6, 2022 (WO) PCTEP2022084490

(51) Int. Cl. D06M 15/15; C07K 14/435; D06M 23/02; D06M 23/06

(52) U.S. Cl. CPC D06M 15/15; C07K 14/43586; D06M 23/02; D06M 23/06; C11D 2111/12; D06M 2200/01; D06M 2200/20; D06M 2200/25; D06M 2200/35; D06M 2200/40; D06M 2200/45; D06M 2200/50

(57) ABSTRACT

The present invention relates to the use of a structural polypeptide for treating or finishing textiles. Specifically, said treating or finishing includes improving or maintaining the properties of textiles such as their optics or introducing properties to textiles. Said treating or finishing also includes restoring textiles.

### CROSS REFERENCE TO RELATED APPLICATIONS

This application is a U.S. National Stage of PCT/EP2022/084490, international filing date Dec. 6, 2022, which claims priority to EP 21212994.4, filing date Dec. 7, 2021.

### REFERENCE TO AN ELECTRONIC SEQUENCE LISTING

The contents of the electronic sequence listing 558-113-PCT-sequence-listing_05-12-2022_ST26.xml; size: 312,158 bytes; and date of creation: Nov. 21, 2023) is herein incorporated by reference in its entirety.

The present invention relates to the use of a structural polypeptide for treating or finishing textiles. Specifically, said treating or finishing includes improving or maintaining the properties of textiles such as their optics or introducing properties to textiles. Said treating or finishing also includes restoring textiles.

### BACKGROUND OF THE INVENTION

It is impossible to imagine life without textiles. They are an important part of our lives. The textile and clothing industry is one of the most important consumer goods sectors. The average life expectancy of textiles varies greatly depending on the nature of the textile. Even after a textile has been used once, a deduction of 10% of the current value is justified. Basically, fashion and aesthetic aspects also influence the life expectancy of textiles.

The intended use may also have to be considered differently here. For example, in the case of commercially textiles—for example, in the hotel and catering sector, or work wear leasing—both the state of preservation as well as the stress caused by frequent washing and cleaning treatments are significantly higher. Textiles used in the bathroom or wellness sector (terry towels, shower towels, bathrobes, washcloths) are particularly affected. Accordingly, identical articles in commercial use have a significantly reduced life expectancy.

The state of preservation, the intended use, the number of washing cycles and the material properties are important factors determining the durability of textiles. In principle, an average life expectancy of a maximum of 200 laundry cycles, often rather 50 laundry cycles, is assumed.

If the life time of textiles is short, they have to be replaced quickly and routinely. The rapidly growing textile industry has become the second largest consumer and polluter of water. It is estimated that the textile industry causes 10 percent of global CO₂ emissions—more than international aviation and maritime shipping combined.

Almost all textiles are pretreated in one or the other way by the manufactures before they are brought to the market. Commercial products that are available for the care of clothing and textiles in the household are well known. These products include substances in liquid, powder and tablet form. Professional services are also available everywhere to treat garments in order to increase their life span. Desired results are typically stain removal, odour elimination, softening, or elimination of static charging and reduced wrinkling. However, these products are often harmful to the environment, and if they are environmentally friendly, they usually are not very effective.

Thus, there is a strong need to extend the life time of textiles, both in the private and commercial sector. Specifically, there is a strong need for substances increasing the life time of textiles which are highly versatile, non-toxic, not environmentally harmful and safe for humans and animals.

The inventors of the present invention surprisingly found that structural polypeptides, such as silk polypeptides, are suitable for treating or finishing textiles. Silk polypeptides are polymers that exhibit exceptional physical properties. Surprisingly, it has now been found that structural polypeptides, such as silk polypeptides, lead to a variety of benefits, including the reduction of graying, the reduction of discoloration or fading of textiles, the improvement of shape retention, or fiber restoration when applied to textiles. Structural polypeptides, such as silk polypeptides, are biocompatible, non-toxic, not polluting and easy to apply and, thus, the ideal substances for textile treating or finishing. The application of these polypeptides, thus, allows the extension of life time of textiles. In this way, environmental pollution can be reduced.

### SUMMARY OF THE INVENTION

The present invention relates to the use of a structural polypeptide, preferably silk polypeptide, for treating or finishing textiles. Particularly, the treating or finishing of textiles includes (i) improving or maintaining the optics of textiles, (ii) improving or maintaining the properties of textiles or introducing properties to textiles, (iii) restoring textiles, (iv) improving the microbiological properties of textiles, and/or (v) improving the olfactory properties of textiles.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The terms “polypeptide” and “protein” are used interchangeably in the context of the present invention. They refer to a long peptide-linked chain of amino acids, e.g. one that is at least 30 amino acids long.

The term “structural polypeptide”, as used herein, refers to any polypeptide which comprises repeat units/repeating building blocks made of amino acids. The structural polypeptide has preferably the ability to perform polypeptide assembly. In particular, the structural polypeptide is capable of forming protein complexes/aggregates in formulations, e.g. hydrogels in aqueous formulations. The structural polypeptide may be selected from the group consisting of a silk polypeptide, keratin, collagen, and elastin or variants or combinations thereof. The structural polypeptide is particularly a recombinant or synthetic structural polypeptide. The structural polypeptide is preferably a (recombinant or synthetic) silk polypeptide, such as a (recombinant or synthetic) spider silk polypeptide. An exemplarily process for producing a silk polypeptide is described in WO 2006/008163 and in WO 2011/120690.

The term “silk polypeptide”, as used herein, refers to a polypeptide which shows, in comparison to other polypeptides, a quite aberrant amino acid composition. In particular, a silk polypeptide possesses large quantities of hydrophobic amino acids such as glycine or alanine. In addition, a silk polypeptide contains highly repetitive amino acid sequences or repetitive units (repeat units, modules), especially in their large core domain.

Based on DNA analysis, it was shown that all silk polypeptide are chains of repetitive units which further comprise a limited set of distinct shorter peptide motifs. The expressions “peptide motif” and “consensus sequence” can be used interchangeably herein. Generally, the silk consensus sequences can be grouped into four major categories: GPGXX, GGX, Aₓ or (GA)ₙ and spacers. These categories of peptide motifs in silk polypeptides have been assigned structural roles. For example, it has been suggested that the GPGXX motif is involved in a R-turn spiral, probably providing elasticity. The GGX motif is known to be responsible for a glycine-rich 31-helix. Both GPGXX and GGX motifs are thought to be involved in the formation of an amorphous matrix that connects crystalline regions, thereby providing elasticity of the fiber. Alanine-rich motifs typically contain 6-9 residues and have been found to form crystalline β-sheets. The spacers typically contain charged groups and separate the iterated peptide motifs into clusters. The silk polypeptide can perform polypeptide assembly. The silk polypeptide is particularly a recombinant or synthetic silk polypeptide. Preferably, the (recombinant or synthetic) silk polypeptide is a (recombinant or synthetic) spider silk polypeptide.

The term “polypeptide assembly”, as used herein, refers to a process in which a disordered system of pre-existing polypeptides forms an organized structure or pattern as a consequence of specific, local interactions (e.g. van der Waals forces, hydrophobic interactions, hydrogen bonds, and/or salt-bridges, etc.) among the polypeptides themselves, without external direction or trigger although external factors might influence speed and nature of polypeptide assembly. This particularly means that when two or more disordered and/or unfolded polypeptides are brought into contact, they interact with each other and consequently form a three dimensional structure. The change from a disordered system to an organized structure or pattern during polypeptide assembly is characterized by a transition from a fluid state to a gelatinous/gel-like and/or solid state and a corresponding increase in viscosity. The transition from a fluid state to a gelatinous/gel-like state can be monitored, for example, by optical measurement or rheology. These techniques are known to the skilled person. The transition from a fluid state to a solid state can be monitored, for example, using optical methods. Preferably, the structural polypeptide conducting polypeptide assembly is a (recombinant or synthetic) silk polypeptide, such as a (recombinant or synthetic) spider silk polypeptide.

The term “polypeptide complexes/aggregates”, as used herein, refers to polypeptide structures which are formed as a consequence or result of polypeptide assembly. In the process of polypeptide assembly, multiple copies/units of polypeptides aggregate into a body or mass without external direction or trigger although external factors might influence speed and nature of polypeptide assembly. In the polypeptide aggregates, the different polypeptides are connected with or attached to each other via covalent (e.g. disulfide bridges) and/or non-covalent interactions (e.g. van der Waals forces, hydrophobic interactions, hydrogen bonds, and/or salt-bridges). It should be clear that a polypeptide aggregate encompasses at least two polypeptides.

In the context of the present invention, the assembly of (recombinant or synthetic) silk polypeptides is described. During the process of silk polypeptide assembly, silk polypeptide complexes/aggregates are formed.

The term “hydrogel”, as used herein, refers to a structure that is formed if the concentration of structural polypeptides is high enough to build a continuous network by which the liquid component is immobilized. Said network is preferably formed by polypeptide assembling of structural polypeptides providing the basis of the hydrogel. In particular, the hydrogel is a hydrophilic polymeric network of structural polypeptides. Said network is stabilized by chemical and/or physical interactions between the structural polypeptides. The network is dispersed throughout an immobilized aqueous phase. The hydrophilicity and stability of the hydrogel permits the penetration and absorption of water (swelling) without dissolving, thus, maintaining its three-dimensional (3D) structure and function.

For example, the concentration of the structural polypeptide, in particular silk polypeptide, in the flowable hydrogel is in the range of 0.1% by weight to 20% by weight, preferably 0.1% by weight to 15% by weight, and more preferably 1% by weight to 10% by weight.
The hydrogel is particularly a flowable hydrogel. Preferably, the hydrogel is a (recombinant or synthetic) silk polypeptide hydrogel. More preferably, the (recombinant or synthetic) silk polypeptide hydrogel is a flowable hydrogel.

The term “flowable hydrogel”, as used herein, refers to a hydrogel that is able/capable of flowing or being flowed. A flowable hydrogel is (still) in a liquid state. The followability of a hydrogel can easily be determined by the skilled person, e.g. by rheology or viscosity measurements. The followability measurements are preferably preformed under standard conditions (25° C.). Alternatively, the hydrogel described/used herein is a non-flowable/solid hydrogel. This hydrogel can be converted to a flowable hydrogel by shear-thinning.

For example, the concentration of the structural polypeptide, in particular silk polypeptide, in the flowable hydrogel is in the range of 0.1% by weight to 20% by weight, preferably 0.1% by weight to 15% by weight, and more preferably 1% by weight to 10% by weight.

The term “textiles”, as used herein, refers to flat, usually two-dimensional, very flexible materials that are created by a textile-forming technique, such as weaving or knitting. Textiles are used in a variety of ways in the clothing industry and in the interior design sector, but also in vehicle and aircraft construction, in medicine, as reinforcement in concrete, lightweight construction and in textile architecture. In particular, the term “textiles”, as used herein, refers to surfaces that consisting mainly of one or more yarns and are produced by a textile technique such as weaving, knitting, warp knitting, etc., either by hand or by machine. The yarn used to make textiles consists of individual fibers of different origins: (i) plant derived natural fibers, such as cotton, linen, coconut, hemp, kapok, ramie, sisal, jute, and/or manila, (ii) animal derived natural fibers, such as wool, silk, angora, cashmere, vikunja, lama, alpaca, camel, mohair, and/or ross hair, (iii) man-made fibers from natural polymers, such as polymers, viscose, cupro, modal, lyocell, acetate, triacetate, and/or polynosic, and/or (iv) man-made fibers from synthetic polymers, such as polyester, polyamide, polyacrylic, elastane, polypropylene, and/or polyurethane. Textiles comprising fibers of any diameter or thread fineness can be used. The textiles may be woven, non-woven, or knitted textiles. The term “textiles”, as used herein, also covers footwear.

The term “treating textiles”, as used herein, refers to any process conducted on textiles that changes, in particular improves, their properties. In particular, the term “treating textiles”, as used herein, refers to any process which leads to higher grade, higher performance textile products. Specifically, the term “treating textiles”, as used herein, includes any process accompanied with the stabilization, cleaning, humidification, consolidation, and/or support of textiles. The stabilization of textiles includes, for example, any treatment that attempts to preserve or reduce the rate of deterioration. Stabilization may include reuniting parts, reinforcing, and/or supporting textiles in order to achieve structural soundness and/or visual completeness.

The term “finishing textiles”, as used herein, refers to any process that converts the textile into a usable material employed. In particular, the term “finishing textiles”, as used herein, includes any process applied to textiles to improve their properties and acceptability. Said process may be performed after textile production, dyeing, drying, or washing. It, thus, covers processes applied to newly produced textiles but also to textiles which are already in use, e.g. in order to increase their life time. Specifically, the term “finishing textiles”, as used herein, includes, any process which improves the appearance or look, the performance, the sizing, the glazing, the touch or hand of textiles. In addition, the term “finishing textiles”, as used herein, includes, any process which adds properties to the textiles in order to enhance their performance. The objective of the various finishing processes is, for example, to make textiles from the loom or knitting frame more acceptable to the consumer. Textiles after leaving the weaving, wrap or knitting machine are sometimes not readily useable. The fabric or cloth, at this stage, contains natural and added impurities. A textile at this stage of manufacture usually goes through a series of processes such as wet processing and finishing. Specifically, finishing includes a broad range of physical and chemical processes/treatments that complete one stage of textile manufacturing, prepare the textile for the next step, and/or make the textile more receptive to the next stage of manufacturing. In any case, finishing adds value to the textile product and makes it more attractive, useful, and functional for the end-user. Improving surface feel, the preservation of properties, and addition of advanced chemical finishes are some examples of textile finishing of textiles which are new or textiles already worn by the consumer.

Especially, the treating or finishing of textiles includes (i) improving or maintaining the optics of textiles, (ii) improving or maintaining the properties of textiles or introducing properties to textiles, (iii) restoring textiles, (iv) improving the microbiological properties of textiles, and/or (v) improving the olfactory properties of textiles.

The present inventors surprisingly found that a structural polypeptide, such as a silk polypeptide, can be used for treating or finishing textiles. Structural polypeptides such as silk polypeptides are biocompatible, non-toxic, not polluting and easy to apply and, thus, the ideal substances to be added to textiles, e.g. in order to increase their life span or to improve their properties before use.

The term “improving or maintaining the optics of textiles”, as used herein, refers to any technique which allows to improve or maintain the appearance of textiles. Factors that make up the visual appearance of textiles are, for example, colourfulness, shining, gloss impression, and/or luminosity.

The fact that dyed fabrics fade from washing and that they turn grey has different causes. It depends on the material and the dyestuff used whether these undesirable effects occur and to which degree.
Many textiles are made from natural cotton fibers. Cotton can be dyed with two different types of dyes: direct dyes and reactive dyes. Direct dyes are the cheaper option. They attach themselves to the cotton fibers, but do not form a real chemical bond with them. Therefore, dyestuff is released from the fabric every time it is washed. The colour of the clothes loses intensity from time to time. The more expensive reactive dyes have so-called reactive groups that enter into a chemical reaction with the cotton (more precisely with its hydroxyl groups, consisting of hydrogen and oxygen). This means that the dyes are firmly bound to the fibers and cannot be dissolved so easily or dissolve only slowly by water.
The greying of dyed fabrics is also observed in textiles made of cotton and other natural fibers. Cotton fabric consists of many short fibers that are twisted together. This also means that there are many fiber ends. When subjected to mechanical stress, such as washing, the fiber ends come to the surface. The surface of the fabric becomes rougher and consequently refracts light differently. The colour changes as a result, losing intensity and brilliance. Modern detergents are supposed to counteract this effect with the help of added chemicals. These chemicals are, however, not always environmentally friendly and sometimes aggressive to skin, which promotes allergies.
The present inventors surprisingly found that a structural polypeptide, such as a silk polypeptide, can be used for improving or maintaining the optics of textiles.

The term “textile restoration, as used herein, refers to any process which helps to prolong the life of textiles. In particular, the term “textile restoration”, as used herein, refers to any process that restores the original textile condition or at least improves the condition of the textile. Specifically, textile restoration includes smoothing of textiles, dissolving fluffs and nodules in textiles, reducing pilling of textiles, reducing fluffing of textiles, and/or filling cavities within the fibers of textiles.

The present inventors surprisingly found that a structural polypeptide, such as a silk polypeptide, is excellent in textile restoration. In this respect, it should be noted that textile restoration also includes textile repair.

Apparel made from textiles can alter skin and its microhabitat, but it can also form a microhabitat in and of itself. The term “improving the microbiological properties of textiles”, as used herein, refers to any process which helps to prevent or reduce the formation of a textile microhabitat. Microbial growth on textiles can cause unpleasant odours, physical irritation, and the loss of tensile strength and decolorization of the fabric. Characteristics of the fabric itself (i.e. woven, non-woven, knitted, thickness, etc.) can also impact moisture and heat retention properties of the fabric, which may in turn affect resident microorganisms. For bacteria, adhesion is the precursor to the colonization of a clothing surface. It precedes proliferation: bacteria that do not adhere to textiles are readily washed off Different bacteria species appear to differ in their ability to adhere to various textile types. For instance, StaphYlococcus spp. were observed to adhere to cotton, polyester and their blends far better than Escherichia coli.

The present inventors surprisingly found that a structural polypeptide, such as a silk polypeptide, is excellent in preventing or at least in reducing textile microhabitat formation.
The mechanism by which the structural polypeptide, such as the silk polypeptide, hinders or at least reduces textile microhabitat formation appears to be the creation of a surface where microorganisms do not readily adhere, live, multiply, and/or colonize.

The term “improving the olfactory properties of textiles”, as used herein, refers to any process which improves the odour of textiles. During use, textile items can develop unpleasant odours that arise from many different sources, both internal and external to the human body. Laundering is not always effective at removing odours, with odour potentially building up over time due to incomplete removal of soils and odorous compounds and/or malodors transferred during the laundering process. Textile odor can lead to consumer dissatisfaction, particularly as there are high expectations that clothing and textile products meet multiple aesthetic and functional needs. The problem of odour in textiles is complex and multi-faceted, with odorous volatile compounds, microorganisms, and precursors to odor, such as sweat, being transferred to, and retained by, fabrics.

The present inventors surprisingly found that a structural polypeptide, such as a silk polypeptide, is excellent in improving the olfactory properties of textiles. The treatment of the textiles with a structural polypeptide, such as a silk polypeptide, is an effective finishing technology to control malodor within textiles. The structural polypeptide, such as a silk polypeptide, is also able to bind and/or protect fragrances or perfumes and, thus, improves the fragrance or perfume impression of textiles or allows the release control of fragrances or perfumes from textiles.

### Embodiments of the Invention

Treating textiles in order to increase their life time and to improve their properties is desirable under environmental aspects. The textile industry causes 10 percent of global CO₂ emissions—more than international aviation and maritime shipping combined.

In addition, textiles after leaving the loom or knitting machine are sometimes not readily useable or acceptable to the consumer. The cloth, at this stage, contains natural and added impurities. A textile at this stage of manufacture usually goes through a series of processes such as wet processing and finishing. Specifically, finishing includes a broad range of physical and chemical processes/treatments that complete one stage of textile manufacturing, prepare the textile for the next step, and/or make the textile more receptive to the next stage of manufacturing. In any case, finishing adds value to the textile product and makes it more attractive, useful, and functional for the end-user. Improving surface feel, the preservation of properties, and addition of advanced chemical finishes are some examples of textile finishing of textiles which are new or textiles already worn by the consumer.

The inventors of the present invention surprisingly found that structural polypeptides, such as silk polypeptides, are suitable for treating or finishing textiles. Silk polypeptides are polymers that exhibit exceptional physical properties. Surprisingly, it has now been found that structural polypeptides, such as silk polypeptides, lead to a variety of benefits, including the reduction of graying, the reduction of discoloration or fading of textiles, the improvement of shape retention, or fiber restoration, when applied to textiles. Structural polypeptides, such as silk polypeptides, are biocompatible, non-toxic, not polluting and easy to apply and, thus, the ideal substances to apply to textiles. The application of these polypeptides, thus, allows the extension of life time of textiles. In this way, environmental pollution can also be reduced.

Thus, in a first aspect, the present invention relates to the use of a structural polypeptide, preferably silk polypeptide, for treating or finishing textiles.

When washing textiles, one sometimes observes that dyed textiles after the washing process create a different colour impression compared to their appearance before the wash. This change in the colour impression of clean textiles can on the one hand be due to the fact that dye components are removed from the textile by the washing process (“fading”). On the other hand, dyes detached from other coloured textiles can be deposited on the textile (“discolouration”). The discolouration aspect can also play a role in the case of undyed textile items when they are treated together with coloured items. Fast resoiling and poor soil removal are also problems that can occur when washing textiles. The “greying” of coloured or white fabrics is also observed if textiles have been washed repeatedly.

The present inventors surprisingly found that the treatment of textiles with structural polypeptides, preferably silk polypeptides, leads to an unexpectedly high colour transfer inhibition. Particularly pronounced is the prevention of the staining of white or other coloured textiles due to dyes washed out of other textiles. The structural polypeptides, preferably silk polypeptides, contribute to both aspects of colour constancy mentioned above, i.e. they reduce both discolouration and fading. In this respect, it should be noted that the change in colour impression does not mean the difference between a dirty and a clean textile, but rather the difference between the clean textile before and after the treatment. Further, the present inventors surprisingly found that textiles which have been treated with structural polypeptides, preferably silk polypeptides, have a higher gloss, luminosity, and whiteness. The typical graying of textiles could be reduced. Reason for this is, among others, that the structural polypeptides, preferably silk polypeptides, smoothens roughened fibers of textiles so that they can reflect the light—so the colours—optimally. Furthermore, the present inventors surprisingly found that textiles treated with structural polypeptides, preferably silk polypeptides, are less prone to resoiling. In addition, the present inventors surprisingly found that soil can easily be removed from textiles that have been treated with structural polypeptides, preferably silk polypeptides.
Thus, in one embodiment, the treating or finishing of textiles includes improving or maintaining the optics of textiles. In one preferred embodiment, the improving or maintaining the optics of textiles encompasses
(i) reducing the graying of textiles,
(ii) improving the whiteness of textiles,
(iii) reducing the discoloration or fading of textiles,
(iv) preventing the change in colour impression of textiles,
(v) improving the gloss impression or shining of textiles,
(vi) improving the luminosity of textiles,
(vii) improving the soil removal of textiles,
(viii) preventing resoiling of textiles, and/or
(ix) reducing the shrinkage of textiles.

The textiles lose over the course of their life time their advantageous properties such as tensile strength, elasticity, shape retention, and mechanical resistance. The textiles literally leaching out over time. Further, the textiles may shrink due to constant washing. Furthermore, the textiles may become brittle and, thus, lose their mechanical resistance. Cotton textiles, for example, consist of many short fibers that are twisted together. This also means that there are many fiber ends. When subjected to mechanical stress in everyday life the fiber ends come to the surface. In addition, textile fibers break due to mechanical stress. Thereby, the mechanical and physical properties of cotton fibers are decreasing.

The present inventors surprisingly found that textiles treated with structural polypeptides, preferably silk polypeptides, have an increased tensile strength and a lower decrease in elasticity compared to textiles not treated with structural polypeptides, preferably silk polypeptides. Further, the present inventors surprisingly found that textiles treated with structural polypeptides, preferably silk polypeptides, retain their shape better and also shrink less compared to textiles not treated with structural polypeptides, preferably silk polypeptides. Furthermore, the present inventors surprisingly found that textiles treated with structural polypeptides, preferably silk polypeptides, have fewer creases/do not crease so easily which in turn facilitates ironing. In addition, the present inventors surprisingly found that textiles treated with structural polypeptides, preferably silk polypeptides, have an improved haptic, e.g. a smoother surface and less or no scratchy surface, compared to textiles not treated with structural polypeptides, preferably silk polypeptides. Moreover, the present inventors surprisingly found that textiles treated with structural polypeptides, preferably silk polypeptides, had different characteristics than before the treatment such as an improved wettability and breathability. They were also able to block UV rays.
Thus, in one another (additional or alternative) embodiment, the treating or finishing of textiles includes improving or maintaining the properties of textiles or introducing properties to textiles. In one preferred embodiment, the improving or maintaining the properties of textiles or introducing properties to textiles encompasses
(i) preventing the loss of tensile strength of textiles,
(ii) preventing the loss of elasticity of textiles,
(iii) improving the shape retention of textiles,
(iv) reducing the shrinkage of textiles,
(v) improving the mechanical resistance of textiles,
(vi) reducing wrinkling/increasing the wrinkling resistance of textiles,
(vii) facilitating ironing of textiles,
(viii) improving the haptics, preferably smoothness, of textiles,
(ix) improving the water vapour permeability of textiles,
(x) improving the wettability of textiles,
(xi) introducing UV ray blocking to textiles,
(xii) improving the breathability of textiles, and/or
(xiii) improving the abrasion resistance of textiles.

Textiles deteriorate over time. Particularly, the fibers of textiles break and/or become rough over time. Exemplarily, cotton textiles consist of many short fibers that are twisted together. This also means that there are many fiber ends. When subjected to mechanical stress in everyday life the fiber ends come to the surface, break, and/or fluff This makes the surface of textiles uneven.

The present inventors surprisingly found that structural polypeptides, preferably silk polypeptides, are able to restore textiles, e.g. smoothing the surface of textiles, dissolving fluffs and nodules in textiles, reducing pilling of textiles, reducing fluffing of textiles, and/or filling cavities within the fibers of textiles.
Thus, in one another (additional or alternative) embodiment, the treating or finishing of textiles includes restoring textiles. In one preferred embodiment, the restoring of textiles encompasses
(i) smoothing (the surface) of textiles,
(ii) dissolving fluffs and nodules in textiles,
(iii) reducing pilling of textiles,
(iv) reducing fluffing of textiles, and/or
(v) filling cavities within the fibers of textiles.

It is well known that textiles can support the growth of bacteria and fungi as they have been found to degrade natural fibers, and to a lesser extent, synthetic fibers. Textile-degrading taxa include the fungal genera Aspergillus. Penicillium and Microsporum and bacterial genera Bacillus, Streptomyces and Pseudomonas. Microbial growth on textiles can cause unpleasant odours, physical irritation, and the loss of tensile strength and decolorization of the fabric. Synthetic fibers are often resistant to microbial attack because microbial enzymes tend to have difficulty breaking their carbon linkages due in part to their hydrophobic nature and poor adsorbing capacity. Biodegradation of synthetic fibers is often facilitated by physical damage, chemical decomposition due to microbial metabolites or enzymatic attacks. By contrast, natural fibers are more susceptible to microbial attack because they tend to have high moisture retention properties and their polymer linkages can be more readily accessed by microbial enzymes, especially after fabric processing in which their protective layers are removed. Additionally, natural fibers can provide nutrients and energy sources for microbes in the form of carbohydrates or proteins. Characteristics of the fabric itself (i.e. woven, non-woven, knitted, thickness, etc.) can also impact moisture and heat retention properties of the fabric, which may in turn affect resident microorganisms. For bacteria, adhesion is the precursor to the colonization of a clothing surface. It precedes proliferation; bacteria that do not adhere to textiles are readily washed off Different bacteria species appear to differ in their ability to adhere to various textile types. For instance, Staphylococcus spp. were observed to adhere to cotton, polyester and their blends far better than Escherichia coli.

The present inventors surprisingly found that structural polypeptides, such as silk polypeptides, are excellent in preventing or at least in reducing textile microhabitat formation. The mechanism by which the structural polypeptides, such as the silk polypeptides, hinder or at least reduce textile microhabitat formation appears to be the creation of a surface where microorganisms do not readily adhere, live, multiply, and/or colonize.
Thus, in one another (additional or alternative) embodiment, the treating or finishing of textiles includes improving the microbiological properties of textiles. In one preferred embodiment, the improving the microbiological properties of textiles encompasses
(i) preventing bad odour of textiles,
(ii) blocking bad odour of textiles, and/or
(iii) preventing or inhibiting microbial growth in textiles.

It is known that textiles lose their initial smell very quickly after washing. They start smelling musty quickly after washing. This makes the wearer feel uncomfortable. The reason for this is that fragrances or perfumes introduced into the textiles during washing are very quickly released into the surrounding air after washing. What remains is the feeling of having to wash the textiles again, even though they are not yet dirty, which damages the fabric and, even worse, pollutes the environment.

The present inventors surprisingly found that textiles treated with structural polypeptides, preferably silk polypeptides, are able to better retain the fragrances or perfumes within their fibers after washing or post treatment. It is assumed that the structural polypeptides, preferably silk polypeptides, put a protective layer aground the fragrances or perfumes so that their good smell is retained longer or bind the fragrances or perfumes so that their good smell escapes more slowly.
Thus, in one another (additional or alternative) embodiment, the treating or finishing of textiles includes improving the olfactory properties of textiles. In one preferred embodiment, the improving the olfactory properties of textiles encompasses
(i) improving the perfume impression of textiles, and/or
(ii) controlling release of perfumes from textiles.

In one more preferred embodiment, the treating or finishing textiles of includes one or more of the following:
improving or maintaining the optics of textiles,
improving or maintaining the properties of textiles or introducing properties to textiles, restoring textiles,
improving the microbiological properties of textiles, and
improving the olfactory properties of textiles.

In one even more preferred embodiment, the treating or finishing textiles includes one or more of the following:
reducing the graying of textiles, improving the whiteness of textiles, reducing the discoloration or fading of textiles, preventing the change in colour impression of textiles, improving the gloss impression or shining of textiles, improving the luminosity of textiles, improving the soil removal of textiles, preventing resoiling of textiles, reducing the shrinkage of textiles, preventing the loss of tensile strength of textiles, preventing the loss of elasticity of textiles, improving the shape retention of textiles, reducing the shrinkage of textiles, improving the mechanical resistance of textiles, reducing wrinkling/increasing the wrinkling resistance of textiles, facilitating ironing of textiles, improving the haptics, preferably smoothness, of textiles, improving the water vapour permeability of textiles, improving the wettability of textiles, introducing UV ray blocking to textiles, improving the breathability of textiles, improving the abrasion resistance of textiles, smoothing of textiles, dissolving fluffs and nodules in textiles, reducing pilling of textiles, reducing fluffing of textiles, filling cavities within the fibers of textiles, preventing bad odour of textiles, blocking bad odour of textiles, preventing or inhibiting microbial growth in textiles, improving the perfume impression of textiles, and controlling release of perfumes from textiles.

In the above described embodiments, the structural polypeptide, preferably silk polypeptide, may be applied directly to the textiles or the structural polypeptide, preferably silk polypeptide, may be applied being part of/comprised in an aqueous formulation to the textiles.

The aqueous formulation may be an aqueous solution, an aqueous suspension, or an aqueous dispersion. The formulation may also have a gel-like structure, e.g. a hydrogel such as a flowable hydrogel. The solvent may be water (H₂O).
It is preferred that the structural polypeptide, preferably silk polypeptide, is comprised in/part of an aqueous formulation.
It is further preferred that the concentration of the structural polypeptide, preferably silk polypeptide, in the aqueous formulation is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.
It is more preferred that the structural polypeptide, preferably silk polypeptide, is comprised in a hydrogel.
It is even more preferred that the concentration of the structural polypeptide, preferably silk polypeptide, in the hydrogel is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.
It is still even more preferred that the structural polypeptide, preferably silk polypeptide, is comprised in a flowable hydrogel.
It is most preferred that the concentration of the structural polypeptide, preferably silk polypeptide, in the flowable hydrogel is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.

In the above-described embodiments, the structural polypeptide used may be a polypeptide which can form polypeptide aggregates. In particular, said polypeptide has the potential to assemble into fibrillary structures (i.e. fibrillary complexes/aggregates of structural polypeptides).

It is preferred that the structural polypeptide is selected from the group consisting of a silk polypeptide, keratin, collagen, and elastin. In particular, the structural polypeptide is a recombinant polypeptide, e.g. a recombinant silk polypeptide, keratin, collagen, or elastin.
It is more preferred that the structural polypeptide is a silk polypeptide, e.g. a recombinant silk polypeptide. The (recombinant) silk polypeptide may be a spider silk polypeptide, e.g. a major ampullate silk polypeptide such as a dragline silk polypeptide, a minor ampullate silk polypeptide, or a flagelliform silk polypeptide of an orb-web spider. Particularly, the silk polypeptide is a spider silk polypeptide, more particularly a recombinant spider silk polypeptide. It is further (alternatively or additionally) more preferred that the silk polypeptide is a polypeptide with an amino acid sequence which comprises or consists of at least 50%, 60%, 65%, 70%, 75%, 80%, 85%, 90%, 95%, or 99% multiple copies of repetitive units or of even 100% multiple copies of repetitive units. Said repetitive units may be identical or different.
It is also (alternatively or additionally) more preferred that the silk polypeptide comprises at least two identical repetitive units. For example, the silk polypeptide comprises between 2 to 96 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 repetitive units. It is also still (alternatively or additionally) more preferred that the silk polypeptide consists of between 40 to 3000 amino acids. It is even more preferred that the silk polypeptide consists of between 40 to 1500 amino acids or between 60 to 1200 amino acids. It is most preferred that the silk polypeptide consists of between 100 to 600 amino acids.
It is even more preferred that the repetitive units are independently selected from module C having an amino acid sequence according to SEQ ID NO: 1 or variants thereof, module C^{Cys} having an amino acid sequence according to SEQ ID NO: 2 or variants thereof, module C^{K} having an amino acid sequence according to SEQ ID NO: 3 or variants thereof, and module C^{LYS} having an amino acid sequence according to SEQ ID NO: 4 or variants thereof.
Module C^{Cys} (SEQ ID NO: 2) is a variant of module C (SEQ ID NO: 1). In this module, the amino acid Ser at position 25 has been replaced by the amino acid Cys. Module C^{K} (SEQ ID NO: 3) is also a variant of module C (SEQ ID NO: 1). Module C^{Lys} (SEQ ID NO: 4) is also a variant of module C (SEQ ID NO: 1). In this module, the amino acid Glu at position 20 has been replaced by the amino acid Lys. In one example, the silk polypeptide comprises 16 C^{K} modules (SEQ ID NO: 3). In one another example, the silk polypeptide comprises 16 modules, wherein the first module (N-terminal) or the last module (C-terminal) is a C^{Lys} module (SEQ ID NO: 4) and the 15 other modules are C modules (SEQ ID NO: 1).
The module C variant differs from the reference module C from which it is derived by up to 1, 2, 3, 4, 5, 6, 7, 8, 9, or 10, 11, 12, 13, 14, or 15 amino acid changes in the amino acid sequence (i.e. substitutions, additions, insertions, deletions, N-terminal truncations and/or C-terminal truncations). Such a module variant can alternatively or additionally be characterized by a certain degree of sequence identity to the reference module from which it is derived. Thus, the module C variant has a sequence identity of at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even 99.9% to the respective reference module C. Preferably, the sequence identity is over a continuous stretch of at least 5, 10, 15, 18, 20, 24, 27, 28, 30, 34, or more amino acids, preferably over the whole length of the respective reference module C.
The sequence identity may be at least 80% over the whole length, may be at least 85% over the whole length, may be at least 90% over the whole length, may be at least 95% over the whole length, may be at least 98% over the whole length, or may be at least 99% over the whole length of the respective reference module C. Alternatively, the sequence identity may be at least 80% over a continuous stretch of at least 5, 10, 15, 18, 20, 24, 28, or 30 amino acids, may be at least 85% over a continuous stretch of at least 5, 10, 15, 18, 20, 24, 28, or 30 amino acids, may be at least 90% over a continuous stretch of at least 5, 10, 15, 18, 20, 24, 28, or 30 amino acids, may be at least 95% over a continuous stretch of at least 5, 10, 15, 18, 20, 24, 28, or 30 amino acids, may be at least 98% over a continuous stretch of at least 5, 10, 15, 18, 20, 24, 28, or 30 amino acids, or may be at least 99% over a continuous stretch of at least 5, 10, 15, 18, 20, 24, 28, or 30 amino acids of the respective reference module C.
A fragment (or deletion) variant of module C has preferably a deletion of up to 1, 2, 3, 4, 5, 6, 7, 8, 9, 10, 11, 12, 13, 14, or 15 amino acids at its N-terminus and/or at its C-terminus. The deletion can also be internally.
Additionally, the module C variant or fragment is only regarded as a module C variant or fragment within the context of the present invention, if the modifications with respect to the amino acid sequence on which the variant or fragment is based do not negatively affect the ability of the silk polypeptide to treat or finish textiles. The skilled person can readily assess whether the silk polypeptide comprising a module C variant or fragment is still capable of treating or finishing textiles. In this respect, it is referred to the examples comprised in the experimental part of the present patent application.
C^{Cys}, C^{K}, or C^{Lys} variants may also be encompassed by the present invention. Regarding the C^{Cys}C^{K}, or C^{Lys} variants, the same explanations/definitions apply which have been made with respect to the module C variant (see above).
It is still even more preferred that the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96.
It is most preferred that the silk polypeptide is selected from the group consisting of C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys}, (C)₃₂C^{Cys}, (C)₄₈C^{Cys}, (C)₂C^{K} (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys}, (C)₆C^{Lys}, (C)₈C^{Lys} (C)₁₆C^{Lys}, (C)₃₂C^{Lys}, (C)₄₈C^{Lys} C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}₂, C^{Cys}₄, C^{Cys}₆, C^{Cys}₈, C^{Cys}₁₆, C^{Cys}₃₂, C^{Cys}₄₈, C^{K}₂, C^{K}₄, C^{K}₆, C^{K}₈, C^{K}₁₆, C^{K}₃₂, C^{K}₄₈, C^{Lys}₂, C^{Lys}₄, C^{Lys}₆, C^{Lys}₈, C^{Lys}₁₆, C^{Lys}₃₂, and C^{Lys}₄₈.
Exemplarily silk polypeptides are the following: The silk polypeptide C₈ (8 times module C) has the amino acid sequence according to SEQ ID NO: 5. The silk polypeptide C₁₆ (16 times module C) has the amino acid sequence according to SEQ ID NO: 6. The silk polypeptide C₃₂ (32 times module C) has the amino acid sequence according to SEQ ID NO: 7. The silk polypeptide C₄₈ (48 times module C) has the amino acid sequence according to SEQ ID NO: 8.
Particularly, the above-described silk polypeptide consists exclusively of repetitive units. In other words, the silk polypeptide particularly does not comprise/is free of non-repetitive units.

The only component that can additionally be present as part of the silk polypeptide is a tag or moiety, e.g. allowing easy transcription of said silk polypeptide in expression systems and/or allowing easy isolation of said silk polypeptide from the expression systems. Said tag may be a his tag or a flag tag.

The aqueous formulation comprising a structural polypeptide, preferably silk polypeptide, may be applied to the textiles via spraying. Alternatively, the aqueous formulation comprising a structural polypeptide, preferably silk polypeptide, may be applied to the textiles via dipping the textiles into the aqueous formulation or by bathing the textiles in the aqueous formulation.

For example, the aqueous formulation comprising a structural polypeptide, preferably silk polypeptide, is applied via spraying as follows: (i) transferring the aqueous formulation comprising a structural polypeptide, preferably silk polypeptide, into a spray can, spraying device, or nebulizer, and (ii) distributing the aqueous formulation comprising the structural polypeptide, preferably silk polypeptide, onto the textile. The textile comprising/having attached thereto the structural polypeptide, preferably silk polypeptide, can further be dried, e.g. at ambient temperature, at room temperature, or at elevated temperatures.
For example, the aqueous formulation comprising a structural polypeptide, preferably silk polypeptide, may be applied via dipping as follows: (i) immersing the textile into the aqueous formulation comprising a structural polypeptide, preferably silk polypeptide, (ii) incubating the textile with the aqueous formulation comprising the structural polypeptide, preferably silk polypeptide, (iii) removing the textile from the aqueous formulation comprising the structural polypeptide, preferably silk polypeptide. The textile comprising/having attached thereto the structural polypeptide, preferably silk polypeptide, can further be dried, e.g. at ambient temperature, at room temperature, or at elevated temperatures.
For example, the aqueous formulation comprising a structural polypeptide, preferably silk polypeptide, may be applied via bathing as follows: (i) introducing the textile into the aqueous formulation comprising a structural polypeptide, preferably silk polypeptide, (ii) bathing the textile in the aqueous formulation comprising the structural polypeptide, preferably silk polypeptide, (iii) removing the textile from the aqueous formulation comprising the structural polypeptide, preferably silk polypeptide. The textile comprising/having attached thereto the structural polypeptide, preferably silk polypeptide, can further be dried, e.g. at ambient temperature, at room temperature, or at elevated temperatures.
In order to achieve excellent results with regard to the treating or finishing of textiles, the textile should be completely sprayed or saturated with the aqueous formulation comprising a structural polypeptide, preferably silk polypeptide.

In case of treating or finishing textiles, in particular for (i) improving or maintaining the optics of textiles, (ii) improving or maintaining the properties of textiles or introducing properties to textiles, (iii) restoring textiles, (iv) improving the microbiological properties of textiles, and/or (v) improving the olfactory properties of textiles, it is preferred that the structural polypeptide, preferably silk polypeptide, is in the form of a hydrogel.

The textile may be any textile for which textile treating of finishing is required. The textiles may be selected from the group consisting of woven, non-woven, or knitted textiles. The (woven or non-woven) textiles may be animal derived textiles, plant derived textiles, synthetic textiles, or blends thereof. Particularly, the animal derived textiles are selected from the group consisting of wool and silk textiles, the plant derived textiles are selected from the group consisting of cotton, flax, rayon, and seaweed textiles, or

the synthetic textiles are selected from the group consisting of nylon, polyester, and spandex textiles.
More particularly, the textile is made of/composed of animal or plant derived fibers, i.e. the textile is an animal derived textile (e.g. wool textile) or a plant derived textile (e.g. cotton textile).

The first aspect of the present invention, as described above, can alternatively be worded as follows: In a first aspect, the present invention relates to a method of using a structural polypeptide for treating or finishing textiles.

In a second aspect, the present invention relates to a method of treating or finishing textiles comprising the steps of:
(i) diluting a dose of an aqueous formulation comprising a structural polypeptide, preferably silk polypeptide, with an aqueous solution to obtain a treating solution, and
(ii) applying the treating solution so formed to the textiles.

The temperature of the aqueous solution is preferably in the range of between 1° C. and 90° C., more preferably in the range of 30° C. and 60° C., and even more preferably in the range of between 40° C. and 60° C., e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90° C.

Preferably, the treating solution is applied by
bathing the textiles in the treating solution,
spraying the treating solution onto the textiles,
nebulizing the treating solution onto the textiles, and/or
dipping the textiles in the treating solution.
   As to the specific bathing, spraying, nebulizing, and dipping techniques, it is referred to the first aspect of the present invention.
   The textile comprising/having attached thereto the structural polypeptide, preferably silk polypeptide, can further be dried, e.g. at ambient temperature, at room temperature, or at elevated temperatures.
   In order to achieve excellent results with regard to the treating or finishing of textiles, the textile should be completely sprayed or saturated with the aqueous formulation comprising a structural polypeptide, preferably silk polypeptide.

The aqueous formulation comprising a structural polypeptide, preferably silk polypeptide, is particularly diluted with the aqueous solution so that the final concentration of the structural polypeptide, preferably silk polypeptide, in the treating solution is in the range of 0.0001% by weight to 15% by weight, specifically 0.1% by weight to 10% by weight, and more specifically 0.1% by weight to 5% by weight, e.g. 0.0001, 0.001, 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15% by weight.

In a third aspect, the present invention relates to a method of treating or finishing textiles comprising the steps of.
(i) providing an aqueous formulation comprising a structural polypeptide, preferably silk polypeptide, and
(ii) applying the aqueous formulation to the textiles.

The temperature of the aqueous formulation is preferably in the range of between 1° C. and 90° C., more preferably in the range of 30° C. and 60° C., and even more preferably in the range of between 40° C. and 60° C., e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90° C.

Preferably, the aqueous formulation is applied by
bathing the textiles in the aqueous formulation,
spraying the aqueous formulation onto the textiles,
nebulizing the aqueous formulation onto the textiles or
dipping the textiles in the aqueous formulation.
   The textile comprising/having attached thereto the structural polypeptide, preferably silk polypeptide, can further be dried, e.g. at ambient temperature, at room temperature, or at elevated temperatures.
   As to the specific bathing, spraying, nebulizing, and dipping techniques, it is referred to the first aspect of the present invention.
   In order to achieve excellent results with regard to the treating or finishing of textiles, the textile should be completely sprayed or saturated with the aqueous formulation comprising a structural polypeptide, preferably silk polypeptide.

The concentration of the structural polypeptide, preferably silk polypeptide, in the aqueous formulation is particularly in the range of 0.01% by weight to 20% by weight, specifically 1% by weight to 15% by weight, and more specifically 1% by weight to 10% by weight, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.

In one embodiment, the treating or finishing of textiles in the method of the second or third aspect includes improving or maintaining the optics of textiles. In one preferred embodiment, the improving or maintaining the optics of textiles encompasses
(i) reducing the graying of textiles,
(ii) improving the whiteness of textiles,
(iii) reducing the discoloration or fading of textiles,
(iv) preventing the change in colour impression of textiles,
(v) improving the gloss impression or shining of textiles,
(vi) improving the luminosity of textiles,
(vii) improving the soil removal of textiles,
(viii) preventing resoiling of textiles, and/or
(ix) reducing the shrinkage of textiles.

In one another (additional or alternative) embodiment, the treating or finishing of textiles in the method of the second or third aspect includes improving or maintaining the properties of textiles or introducing properties to textiles. In one preferred embodiment, the improving or maintaining the properties of textiles or introducing properties to textiles encompasses
(i) preventing the loss of tensile strength of textiles,
(ii) preventing the loss of elasticity of textiles,
(iii) improving the shape retention of textiles,
(iv) reducing the shrinkage of textiles,
(v) improving the mechanical resistance of textiles,
(vi) reducing wrinkling/increasing the wrinkling resistance of textiles,
(vii) facilitating ironing of textiles,
(viii) improving the haptics, preferably smoothness, of textiles,
(ix) improving the water vapour permeability of textiles,
(x) improving the wettability of textiles,
(xi) introducing UV ray blocking to textiles, and/or
(xii) improving the breathability of textiles, and/or
(xiii) improving the abrasion resistance of textiles.

In one another (additional or alternative) embodiment, the treating or finishing of textiles in the method of the second or third aspect includes restoring textiles. In one preferred embodiment, the restoring of textiles encompasses
(i) smoothing of textiles,
(ii) dissolving fluffs and nodules in textiles,
(iii) reducing pilling of textiles,
(iv) reducing fluffing of textiles, and/or
(v) filling cavities within the fibers of textiles.

In one another (additional or alternative) embodiment, the treating or finishing of textiles in the method of the second or third aspect includes improving the microbiological properties of textiles. In one preferred embodiment, the microbiological properties of textiles encompasses
(i) preventing bad odour of textiles,
(ii) blocking bad odour of textiles, and/or
(iii) preventing or inhibiting microbial growth in textiles.

In one another (additional or alternative) embodiment, the treating or finishing of textiles in the method of the second or third aspect includes improving the olfactory properties of textiles. In one preferred embodiment, the improving the olfactory properties of textiles encompasses
(i) improving the perfume impression of textiles, and/or
(ii) controlling release of perfumes from textiles.

In one more preferred embodiment, the treating or finishing of textiles includes one or more of the following:
improving or maintaining the optics of textiles,
improving or maintaining the properties of textiles or introducing properties to textiles, restoring textiles,
improving the microbiological properties of textiles, and
improving the olfactory properties of textiles.

In one even more preferred embodiment, the treating or finishing of textiles includes one or more of the following:
reducing the graying of textiles, improving the whiteness of textiles, reducing the discoloration or fading of textiles, preventing the change in colour impression of textiles, improving the gloss impression or shining of textiles, improving the luminosity of textiles, improving the soil removal of textiles, preventing resoiling of textiles, reducing the shrinkage of textiles, preventing the loss of tensile strength of textiles, preventing the loss of elasticity of textiles, improving the shape retention of textiles, reducing the shrinkage of textiles, improving the mechanical resistance of textiles, reducing wrinkling/increasing the wrinkling resistance of textiles, facilitating ironing of textiles, improving the haptics, preferably smoothness, of textiles, improving the water vapour permeability of textiles, improving the wettability of textiles, introducing UV ray blocking to textiles, improving the breathability of textiles, improving the abrasion resistance of textiles, smoothing of textiles, dissolving fluffs and nodules in textiles, reducing pilling of textiles, reducing fluffing of textiles, filling cavities within the fibers of textiles, preventing bad odour of textiles, blocking bad odour of textiles, preventing or inhibiting microbial growth in textiles, improving the perfume impression of textiles, and controlling release of perfumes from textiles.

The aqueous formulation described in the second or third aspect may be an aqueous solution, an aqueous suspension, or an aqueous dispersion. The formulation may also have a gel-like structure, e.g. a hydrogel. The solvent may be water (H₂O).

It is preferred that the structural polypeptide, preferably silk polypeptide, is comprised in/part of an aqueous formulation.
It is further preferred that the concentration of the structural polypeptide, preferably silk polypeptide, in the aqueous formulation is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.
It is more preferred that the structural polypeptide, preferably silk polypeptide, is comprised in a hydrogel.
It is even more preferred that the concentration of the structural polypeptide, preferably silk polypeptide, in the hydrogel is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.
It is still even more preferred that the structural polypeptide, preferably silk polypeptide, is comprised in a flowable hydrogel.
It is most preferred that the concentration of the structural polypeptide, preferably silk polypeptide, in the flowable hydrogel is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.
In order to achieve excellent results with regard to the treating or finishing of textiles, the textile should be completely sprayed or saturated with the aqueous formulation comprising a structural polypeptide, preferably silk polypeptide.

The structural polypeptide used in the method of the second or third aspect may be a polypeptide which can form polypeptide aggregates. In particular, said polypeptide has the potential to assemble into fibrillary structures (i.e. fibrillary complexes of structural polypeptides).

It is preferred that the structural polypeptide is selected from the group consisting of a silk polypeptide, keratin, collagen, and elastin. In particular, the structural polypeptide is a recombinant polypeptide, e.g. a recombinant silk polypeptide, keratin, collagen, or elastin.
It is more preferred that the structural polypeptide is a silk polypeptide, e.g. a recombinant silk polypeptide. The (recombinant) silk polypeptide may be a spider silk polypeptide, e.g. a major ampullate silk polypeptide such as a dragline silk polypeptide, a minor ampullate silk polypeptide, or a flagelliform silk polypeptide of an orb-web spider. Particularly, the silk polypeptide is a spider silk polypeptide, more particularly a recombinant spider silk polypeptide.
It is further (alternatively or additionally) more preferred that the silk polypeptide is a polypeptide with an amino acid sequence which comprises or consists of at least 50%, 60%, 65%, 70%, 75%, 80%, 85%, 90%, 95%, or 99% multiple copies of repetitive units or of even 100% multiple copies of repetitive units. Said repetitive units may be identical or different.
It is also (alternatively or additionally) more preferred that the silk polypeptide comprises at least two identical repetitive units. For example, the silk polypeptide comprises between 2 to 96 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 repetitive units.
It is also still (alternatively or additionally) more preferred that the silk polypeptide consists of between 40 to 3000 amino acids. It is even more preferred that the silk polypeptide consists of between 40 to 1500 amino acids or between 60 to 1200 amino acids. It is most preferred that the silk polypeptide consists of between 100 to 600 amino acids.
It is even more preferred that the repetitive units are independently selected from module C having an amino acid sequence according to SEQ ID NO: 1 or variants thereof, module C^{Cys} having an amino acid sequence according to SEQ ID NO: 2 or variants thereof, module C^{K} having an amino acid sequence according to SEQ ID NO: 3 or variants thereof, and module C^{Lys} having an amino acid sequence according to SEQ ID NO: 4 or variants thereof.
Module C^{Cys} (SEQ ID NO: 2) is a variant of module C (SEQ ID NO: 1). In this module, the amino acid Ser at position 25 has been replaced by the amino acid Cys. Module C^{K} (SEQ ID NO: 3) is also a variant of module C (SEQ ID NO: 1). Module C^{Lys} (SEQ ID NO: 4) is also a variant of module C (SEQ ID NO: 1). In this module, the amino acid Glu at position 20 has been replaced by the amino acid Lys. In one example, the silk polypeptide comprises 16 C^{K} modules (SEQ ID NO: 3). In one another example, the silk polypeptide comprises 16 modules, wherein the first module (N-terminal) or the last module (C-terminal) is a C^{Lys} module (SEQ ID NO: 4) and the 15 other modules are C modules (SEQ ID NO: 1).
As to the module C, C^{Cys}, C^{K}, or C^{Lys} variants, it is referred to the first aspect of the present invention.
It is still even more preferred that the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96.

It is most preferred that the silk polypeptide is selected from the group consisting of C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys}, (C)₃₂C^{Cys}, (C)₄₈C^{Cys}, (C)₂C^{K}, (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys}, (C)₆C^{Lys}, (C)₈C^{Lys}, (C)₁₆C^{Lys}, (C)₃₂C^{Lys}, (C)₄₈C^{Lys}, C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}₂, C^{Cys}₄, C^{Cys}₆, C^{Cys}₈, C^{Cys}₁₆, C^{Cys}₃₂, C^{Cys}₄₈, C^{K}₂, C^{K}₄, C^{K}₆, C^{K}₈, C^{K}₁₆, C^{K}₃₂, C^{K}₄₈, C^{Lys}₂, C^{Lys}₄, C^{Lys}₆, C^{Lys}₈, C^{Lys}₁₆, C^{Lys}₃₂, and C^{Lys}₄₈.

Exemplarily silk polypeptides are the following: The silk polypeptide C₈ (8 times module C) has the amino acid sequence according to SEQ ID NO: 5. The silk polypeptide C₁₆ (16 times module C) has the amino acid sequence according to SEQ ID NO: 6. The silk polypeptide C₃₂ (32 times module C) has the amino acid sequence according to SEQ ID NO: 7. The silk polypeptide C₄₈ (48 times module C) has the amino acid sequence according to SEQ ID NO: 8.
Particularly, the above described silk polypeptide consists exclusively of repetitive units. In other words, the silk polypeptide particularly does not comprise/is free of non-repetitive units. The only component that can additionally be present as part of the silk polypeptide is a tag or moiety, e.g. allowing easy transcription of said silk polypeptide in expression systems and/or allowing easy isolation of said silk polypeptide from the expression systems. Said tag may be a his tag or a flag tag.

In case of treating or finishing textiles, in particular for (i) improving or maintaining the optics of textiles, (ii) improving or maintaining the properties of textiles or introducing properties to textiles, (iii) restoring textiles, (iv) improving the microbiological properties of textiles, and/or (v) improving the olfactory properties of textiles, it is preferred that the structural polypeptide, preferably silk polypeptide, is in the form of a hydrogel.

In case of increasing the olfactory properties of textiles, the structural polypeptide, preferably silk polypeptide, it is preferred that the structural polypeptide, preferably silk polypeptide, is in the form of a capsule, e.g. a silk polypeptide capsule having a silk polypeptide shell and having perfume and/or fragrances encapsulated therein.

The textile may be any textile for which textile treating of finishing is required. The textiles may be selected from the group consisting of woven, non-woven, or knitted textiles. The (woven or non-woven) textiles may be animal derived textiles, plant derived textiles, synthetic textiles, or blends thereof. Particularly,

the animal derived textiles are selected from the group consisting of wool and silk textiles, the plant derived textiles are selected from the group consisting of cotton, flax, rayon, and seaweed textiles, or
the synthetic textiles are selected from the group consisting of nylon, polyester, and spandex textiles.
More particularly, the textile is made of/composed of animal or plant derived fibers, i.e. the textile is an animal derived textile (e.g. wool textile) or a plant derived textile (e.g. cotton textile).

It is specifically preferred that the structural polypeptide, preferably silk polypeptide, referred to in the first to third aspect of the present invention is comprised in/part of a laundry detergent composition or fabric softener composition. It is more specifically preferred that the aqueous formulation comprising the structural polypeptide, preferably silk polypeptide, referred to in the first to third aspect of the present invention is comprised in/part of a laundry detergent composition or fabric softener composition. It is even more specifically preferred that the (flowable) hydrogel comprising the structural polypeptide, preferably silk polypeptide, referred to in the first to third aspect of the present invention is comprised in/part of a laundry detergent composition or fabric softener composition.

Accordingly, the present invention relates, in a further aspect, to a laundry detergent composition comprising a structural polypeptide, preferably silk polypeptide, or to a fabric softener composition comprising a structural polypeptide, preferably silk polypeptide. As to the specific embodiments with respect to the structural polypeptide, preferably silk polypeptide, it is referred to the first to third aspect of the present invention.

In addition to ingredients that are indispensable for the washing process, such as surfactants and builder materials, laundry detergent compositions further comprise active ingredients like foam regulators, inhibitors, bleaching agents, bleach activators, and/or enzymes. Said active ingredients contribute to a variety of desirable properties of detergents.

The present inventors surprisingly found that a structural polypeptide, preferably silk polypeptide, is such an active ingredient having diverse properties helpful in the process of treating or finishing textiles. Thus, as part of a laundry detergent composition or fabric softener composition, the structural polypeptide, preferably silk polypeptide, can be used for treating or finishing textiles. Specifically, the structural polypeptide, preferably silk polypeptide, can be used for (i) improving or maintaining the optics of textiles, (ii) improving or maintaining the properties of textiles or introducing properties to textiles, (iii) restoring textiles, (iv) improving the microbiological properties of textiles, and/or (v) improving the olfactory properties of textiles. As to the specific uses, it is further referred to the first to third aspect of the prevent invention.

In a fourth aspect, the present invention relates to a kit comprising
(i) a laundry detergent composition, and/or
(ii) a fabric softener composition, and
(iii) a structural polypeptide, preferably silk polypeptide.
   For example, the kit comprises (i) a laundry detergent composition and a structural polypeptide, preferably silk polypeptide, (ii) a fabric softener composition and a structural polypeptide, preferably silk polypeptide, or (iii) a laundry detergent composition, a fabric softener composition, and a structural polypeptide, preferably silk polypeptide.

The kit comprising
(i) a laundry detergent composition, and/or
(ii) a fabric softener composition, and
(iii) a structural polypeptide, preferably silk polypeptide, may optionally further comprise a textile treatment composition and/or a textile finishing composition.
   For example, the kit comprises (i) a laundry detergent composition, a structural polypeptide, preferably silk polypeptide, and a textile treatment composition, (ii) a fabric softener composition, a structural polypeptide, preferably silk polypeptide, and a textile treatment composition, or (iii) a laundry detergent composition, a fabric softener composition, a structural polypeptide, preferably silk polypeptide, and a textile treatment composition.
   Alternatively, the kit comprises (i) a laundry detergent composition, a structural polypeptide, preferably silk polypeptide, and a textile finishing composition (ii) a fabric softener composition, a structural polypeptide, preferably silk polypeptide, and a textile finishing composition, or (iii) a laundry detergent composition, a fabric softener composition, a structural polypeptide, preferably silk polypeptide, and a textile finishing composition.
   Specifically, the kit comprises (i) a laundry detergent composition, a structural polypeptide, preferably silk polypeptide, a textile treatment composition, and a textile finishing composition (ii) a fabric softener composition, a structural polypeptide, preferably silk polypeptide, a textile treatment composition, and a textile finishing composition, or (iii) a laundry detergent composition, a fabric softener composition, a structural polypeptide, preferably silk polypeptide, a textile treatment composition, and a textile finishing composition.
   The textile treatment composition as mentioned above is preferably an impregnation spray and/or the textile finishing composition as mentioned above is preferably a composition rendering the textile water repellent.

The structural polypeptide, preferably silk polypeptide, may be applied directly to the textiles or the structural polypeptide, preferably silk polypeptide, may be applied being part of/comprised in an aqueous formulation to the textiles. The aqueous formulation may be an aqueous solution, an aqueous suspension, or an aqueous dispersion. The formulation may also have a gel-like structure, e.g. a hydrogel. The solvent may be water (H₂O).

It is preferred that the structural polypeptide, preferably silk polypeptide, is comprised in/part of an aqueous formulation.
It is further preferred that the concentration of the structural polypeptide, preferably silk polypeptide, in the aqueous formulation is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.
It is more preferred that the structural polypeptide, preferably silk polypeptide, is comprised in a hydrogel.
It is even more preferred that the concentration of the structural polypeptide, preferably silk polypeptide, in the hydrogel is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.
It is still even more preferred that the structural polypeptide, preferably silk polypeptide, is comprised in a flowable hydrogel.
It is most preferred that the concentration of the structural polypeptide, preferably silk polypeptide, in the flowable hydrogel is in the range of 1% by weight to 20% by weight, specifically 1% by weight to 15% by weight, and more specifically 1% by weight to 10% by weight, e.g. 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% by weight.
In order to achieve excellent results with regard to the treating or finishing of textiles, the textile should be completely sprayed or saturated with the aqueous formulation comprising a structural polypeptide, preferably silk polypeptide.

As to the specific embodiments of the structural polypeptide, preferably silk polypeptide, it is referred to the first to third aspect of the present invention.

Particularly, the above described silk polypeptide consists exclusively of repetitive units. In other words, the silk polypeptide particularly does not comprise/is free of non-repetitive units. The only component that can additionally be present as part of the silk polypeptide is a tag or moiety, e.g. allowing easy transcription of said silk polypeptide in expression systems and/or allowing easy isolation of said silk polypeptide from the expression systems. Said tag may be a his tag or a flag tag.

The kit is preferably used for treating or finishing textiles. Especially, the fibers or textiles are treated with the composition, e.g. liquid or nebulized liquid composition, to improve their properties (see below). The treatment can be carried out before final fiber/textile completion or after final fiber/textile generation.

In one embodiment, the treating or finishing of textiles includes improving or maintaining the optics of textiles. In one preferred embodiment, the improving or maintaining the optics of textiles encompasses
(i) reducing the graying of textiles,
(ii) improving the whiteness of textiles,
(iii) reducing the discoloration or fading of textiles,
(iv) preventing the change in colour impression of textiles,
(v) improving the gloss impression or shining of textiles,
(vi) improving the luminosity of textiles,
(vii) improving the soil removal of textiles,
(viii) preventing resoiling of textiles, and/or
(ix) reducing the shrinkage of textiles.

In one another (additional or alternative) embodiment, the treating or finishing of textiles includes improving or maintaining the properties of textiles or introducing properties to textiles.

In one preferred embodiment, the improving or maintaining the properties of textiles or introducing properties to textiles encompasses
(i) preventing the loss of tensile strength of textiles,
(ii) preventing the loss of elasticity of textiles,
(iii) improving the shape retention of textiles,
(iv) reducing the shrinkage of textiles,
(v) improving the mechanical resistance of textiles,
(vi) reducing wrinkling/increasing the wrinkling resistance of textiles,
(vii) facilitating ironing of textiles,
(viii) improving the haptics, preferably smoothness, of textiles,
(ix) improving the water vapour permeability of textiles,
(x) improving the wettability of textiles,
(xi) introducing UV ray blocking to textiles,
(xii) improving the breathability of textiles, and/or
(xiii) improving the abrasion resistance of textiles.

In one another (additional or alternative) embodiment, the treating or finishing of textiles includes restoring textiles. In one preferred embodiment, the restoring of textiles encompasses
(i) smoothing of textiles,
(ii) dissolving fluffs and nodules in textiles,
(iii) reducing pilling of textiles,
(iv) reducing fluffing of textiles, and/or
(v) filling cavities within the fibers of textiles.

In one another (additional or alternative) embodiment, the treating or finishing of textiles includes improving the microbiological properties of textiles. In one preferred embodiment, the microbiological properties of textiles encompasses
(i) preventing bad odour of textiles,
(ii) blocking bad odour of textiles, and/or
(iii) preventing or inhibiting microbial growth in textiles.

In one another (additional or alternative) embodiment, the treating or finishing of textiles includes improving the olfactory properties of textiles. In one preferred embodiment, the improving the olfactory properties of textiles encompasses
(i) improving the perfume impression of textiles, and/or
(ii) controlling release of perfumes from textiles.

In one more preferred embodiment, the treating or finishing of textiles includes one or more of the following:
improving or maintaining the optics of textiles,
improving or maintaining the properties of textiles or introducing properties to textiles, restoring textiles,
improving the microbiological properties of textiles, and
improving the olfactory properties of textiles.

In one even more preferred embodiment, the treating or finishing of textiles includes one or more of the following:

reducing the graying of textiles, improving the whiteness of textiles, reducing the discoloration or fading of textiles, preventing the change in colour impression of textiles, improving the gloss impression or shining of textiles, improving the luminosity of textiles, improving the soil removal of textiles, preventing resoiling of textiles, reducing the shrinkage of textiles, preventing the loss of tensile strength of textiles, preventing the loss of elasticity of textiles, improving the shape retention of textiles, reducing the shrinkage of textiles, improving the mechanical resistance of textiles, reducing wrinkling/increasing the wrinkling resistance of textiles, facilitating ironing of textiles, improving the haptics, preferably smoothness, of textiles, improving the water vapour permeability of textiles, improving the wettability of textiles, introducing UV ray blocking to textiles, improving the breathability of textiles, improving the abrasion resistance of textiles, smoothing of textiles, dissolving fluffs and nodules in textiles, reducing pilling of textiles, reducing fluffing of textiles, filling cavities within the fibers of textiles, preventing bad odour of textiles, blocking bad odour of textiles, preventing or inhibiting microbial growth in textiles, improving the perfume impression of textiles, and controlling release of perfumes from textiles.

The invention is summarized as follows:

1. Use of a structural polypeptide for treating or finishing textiles.
2. The use of item 1, wherein treating or finishing textiles includes improving or maintaining the optics of textiles.
3. The use of item 2, wherein improving or maintaining the optics of textiles encompasses
(i) reducing the graying of textiles,
(ii) improving the whiteness of textiles,
(iii) reducing the discoloration or fading of textiles,
(iv) preventing the change in colour impression of textiles,
(v) improving the gloss impression or shining of textiles,
(vi) improving the luminosity of textiles,
(vii) improving the soil removal of textiles,
(viii) preventing resoiling of textiles, and/or
(ix) reducing the shrinkage of textiles.
   4. The use of any one of items 1 to 3, wherein treating or finishing textiles includes improving or maintaining the properties of textiles or introducing properties to textiles.
   5. The use of item 4, wherein improving or maintaining the properties of textiles or introducing properties to textiles encompasses
(i) preventing the loss of tensile strength of textiles,
(ii) preventing the loss of elasticity of textiles,
(iii) improving the shape retention of textiles,
(iv) reducing the shrinkage of textiles,
(v) improving the mechanical resistance of textiles,
(vi) reducing wrinkling/increasing the wrinkling resistance of textiles,
(vii) facilitating ironing of textiles,
(viii) improving the haptics, preferably smoothness, of textiles,
(ix) improving the water vapour permeability of textiles,
(x) improving the wettability of textiles,
(xi) introducing UV ray blocking to textiles, and/or
(xii) improving the breathability of textiles, and/or
(xiii) improving the abrasion resistance of textiles.
   6. The use of any one of items 1 to 5, wherein treating or finishing textiles includes restoring textiles.
   7. The use of item 6, wherein restoring textiles encompasses
(i) smoothing of textiles,
(ii) dissolving fluffs and nodules in textiles,
(iii) reducing pilling of textiles,
(iv) reducing fluffing of textiles, and/or
(v) filling cavities within the fibers of textiles.
   8. The use of any one of items 1 to 7, wherein treating or finishing textiles includes improving the microbiological properties of textiles.
   9. The use of item 8, wherein improving the microbiological properties of textiles encompasses
(i) preventing bad odour of textiles,
(ii) blocking bad odour of textiles, and/or
(iii) preventing or inhibiting microbial growth in textiles.
   10. The use of any one of items 1 to 9, wherein treating or finishing textiles includes improving the olfactory properties of textiles.
   11. The use of item 10, wherein improving the olfactory properties of textiles encompasses
(i) improving the perfume impression of textiles, and/or
(ii) controlling release of perfumes from textiles.
   12. The use of any one of items 1 to 11, wherein the structural polypeptide is comprised in an aqueous formulation.
   13. The use of item 12, wherein the concentration of the structural polypeptide in the aqueous formulation is in the range of 0.01% by weight to 20% by weight, preferably 0.1% by weight to 15% by weight, and more preferably 1% by weight to 10% by weight.
   14. The use of items 12 or 13, wherein the aqueous formulation has a gel-like structure.
   15. The use of item 14, wherein the gel-like structure is a hydrogel.
   16. The use of any one of items 1 to 15, wherein the structural polypeptide is a silk polypeptide.
   17. The use of item 16, wherein the silk polypeptide is a recombinant silk polypeptide.
   18. The use of items 16 or 17, wherein the silk polypeptide comprises or consists of (at least two identical) repetitive units.
   19. The use of any one of items 16 to 18, wherein the silk polypeptide does not comprise/is free of non-repetitive units.
   20. The use of items 18 or 19, wherein the repetitive units are independently selected from the group consisting of module C having an amino acid sequence according to SEQ ID NO: 1 or variants thereof, module C^{Cys} having an amino acid sequence according to SEQ ID NO: 2 or variants thereof, module C^{K} having an amino acid sequence according to SEQ ID NO: 3 or variants thereof, and module C^{Lys} having an amino acid sequence according to SEQ ID NO: 4 or variants thereof.
   21. The use of any one of items 16 to 20, wherein the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96.
   22. The use of item 21, wherein the silk polypeptide is selected from the group consisting of C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys}, (C)₃₂C^{Cys}, (C)₄₈C^{Cys}, (C)₂C^{K}, (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys}, (C)₆C^{Lys}, (C)₈C^{Lys}, (C)₁₆C^{Lys}, (C)₃₂C^{Lys}, (C)₄₈C^{Lys}, C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}, C^{Cys}₄, C^{Cys}₆, C^{Cys}₈, C^{Cys}₁₆, C^{Cys}₃₂, C^{Cys}₄₈, C^{K}₂, C^{K}₄, C^{K}₆, C^{K}₈, C^{K}₁₆, C^{K}₃₂, C^{K}₄₈, C^{Lys}₂, C^{Lys}₄, C^{Lys}₆, C^{Lys}₈, C^{Lys}₁₆, C^{Lys}₃₂, and C^{Lys}₄₈.
   23. The use of any one of items 1 to 22, wherein the textiles are selected from the group consisting of woven, non-woven, or knitted textiles.
   24. The use of any one of items 1 to 23, wherein the textiles are animal derived textiles, plant derived textiles, synthetic textiles, or blends thereof.
   25. The use of item 24, wherein
the animal derived textiles are selected from the group consisting of wool and silk textiles,
the plant derived textiles are selected from the group consisting of cotton, flax, rayon, and seaweed textiles, or
the synthetic textiles are selected from the group consisting of nylon, polyester, and spandex textiles.
   26. A method of treating or finishing textiles comprising the steps of:
(i) diluting a dose of an aqueous formulation comprising a structural polypeptide with water to obtain a treating solution, and
(ii) applying the treating solution so formed to the textiles.
   27. The method of item 26, wherein the application of the treating solution takes place by bathing the textiles in the treating solution, spraying the treating solution onto the textiles, nebulizing the treating solution onto the textiles, or dipping the textiles in the treating solution.
   28. A method of treating or finishing textiles comprising the steps of:
(i) providing an aqueous formulation comprising a structural polypeptide, and
(ii) applying the aqueous formulation to the textiles.
   29. The method of item 28, wherein the application of the aqueous formulation takes place by
bathing the textiles in the aqueous formulation,
spraying the aqueous formulation onto the textiles,
nebulizing the aqueous formulation onto the textiles or
dipping the textiles in the aqueous formulation.
   30. The method of any one of items 26 to 29, wherein the concentration of the structural polypeptide in the aqueous formulation is in the range of 0.01% by weight to 20% by weight, preferably 0.10% by weight to 150% by weight, and more preferably 1T % by weight to 10% by weight.
   31. The method of any one of items 26 to 30, wherein the aqueous formulation has a gel-like structure.
   32. The method of item 31, wherein the gel-like structure is a hydrogel.
   33. The method of any one of items 26 to 32, wherein the structural polypeptide is a silk polypeptide.
   34. The method of item 33, wherein the silk polypeptide is a recombinant silk polypeptide.
   35. The method of items 33 or 34, wherein the silk polypeptide comprises or consists of (at least two identical) repetitive units.
   36. The method of any one of items 33 to 35, wherein the silk polypeptide does not comprise/is free of non-repetitive units.
   37. The method of items 35 or 36, wherein the repetitive units are independently selected from the group consisting of module C having an amino acid sequence according to SEQ ID NO: 1 or variants thereof, module C^{Cys} having an amino acid sequence according to SEQ ID NO: 2 or variants thereof, module C^{K} having an amino acid sequence according to SEQ ID NO: 3 or variants thereof, and module C^{Lys} having an amino acid sequence according to SEQ ID NO: 4 or variants thereof.
   38. The method of any one of items 33 to 37, wherein the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96.
   39. The method of item 38, wherein the silk polypeptide is selected from the group consisting of C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys} (C)₃₂C^{Cys}, (C)₄₈C^{Cys}, (C)₂C^{K}, (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys}, (C)₆C^{Lys}, (C)₈C^{Lys}, (C)₁₆C^{Lys}, (C)₃₂C^{Lys}, (C)₄₈C^{Lys} C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}₂, C^{Cys}₄, C^{Cys}₆, C^{Cys}₈, C^{Cys}₁₆, C^{Cys}₃₂, C^{Cys}₄₈, C^{K}₂, C^{K}₄, C^{K}₆, C^{K}₈, c^{K}₁₆, C^{K}₃₂, C^{K}₄₈, C^{Lys}₂, C^{Lys}₄, C^{Lys}₆, C^{Lys}₈, C^{Lys}₁₆, C^{Lys}₃₂, and C^{Lys}₄₈.
   40. The method of any one of items 26 to 39, wherein the textiles are selected from the group consisting of woven, non-woven, or knitted textiles.
   41. The method of any one of items 26 to 40, wherein the textiles are animal derived textiles, plant derived textiles, synthetic textiles, or blends thereof.
   42. The method of item 41, wherein
the animal derived textiles are selected from the group consisting of wool and silk textiles,
the plant derived textiles are selected from the group consisting of cotton, flax, rayon, and seaweed textiles, or
the synthetic textiles are selected from the group consisting of nylon, polyester, and spandex textiles.
   43. A kit comprising
(i) a laundry detergent composition, and/or
(ii) a fabric softener composition, and
(iii) a structural polypeptide.
   44. The kit of item 43, wherein the structural polypeptide is comprised in an aqueous formulation.
   45. The kit of items 43 or 44, wherein the concentration of the structural polypeptide in the formulation is in the range of 0.001% by weight to 20% by weight, preferably 01% by weight to 10% by weight, and more preferably 0.01% by weight to 5% by weight.
   46. The kit of items 44 or 45, wherein the aqueous formulation has a gel-like structure.
   47. The kit of item 46, wherein the gel-like structure is a hydrogel.
   48. The kit of any one of items 43 to 47, wherein the structural polypeptide is a silk polypeptide.
   49. The kit of item 48, wherein the silk polypeptide is a recombinant silk polypeptide.
   50. The kit of items 48 or 49, wherein the silk polypeptide comprises or consists of (at least two identical) repetitive units.
   51. The kit of any one of items 48 to 50, wherein the silk polypeptide does not comprise/is free of non-repetitive units.
   52. The kit of items 50 or 51, wherein the repetitive units are independently selected from the group consisting of module C having an amino acid sequence according to SEQ ID NO:
   1 or variants thereof, module C^{Cys} having an amino acid sequence according to SEQ ID NO: 2 or variants thereof, module C^{K} having an amino acid sequence according to SEQ ID NO: 3 or variants thereof, and module C^{Lys} having an amino acid sequence according to SEQ ID NO: 4 or variants thereof.
   53. The kit of any one of items 48 to 52, wherein the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96.
   54. The kit of item 53, wherein the silk polypeptide is selected from the group consisting of C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys}, (C)₃₂C^{Cys} (C)₄₈C^{Cys}, (C)₂C^{K}, (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys} (C)₆C^{Lys}, (C)₈C^{Lys}, (C)₁₆C^{Lys}, (C)₃₂C^{Lys}, (C)₄₈C^{Lys} C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}₂, C^{Cys}₄, C^{Cys}₆, C^{Cys}₈, C^{CYs}₁₆, C^{Cys}₃₂, C^{Cys}₄₈, C^{K}₂, C^{K}₄, C^{K}₆, C^{K}₈, C^{K}₁₆, C^{K}₃₂, C^{K}₄₈, C^{LYS}₂, C^{LYS}₄, C^{LYS}₆, C^{LYS}₈, C^{LYS}₁₆, C^{LYS}₃₂, and C^{LYS}₄₈.

The invention is further summarized as follows:

1. Use of a structural polypeptide for treating or finishing textiles.
2. The use of item 1, wherein treating or finishing textiles includes improving or maintaining the optics of textiles.
3. The use of item 2, wherein improving or maintaining the optics of textiles encompasses
(i) reducing the graying of textiles,
(ii) improving the whiteness of textiles,
(iii) reducing the discoloration or fading of textiles,
(iv) preventing the change in colour impression of textiles,
(v) improving the gloss impression or shining of textiles,
(vi) improving the luminosity of textiles,
(vii) improving the soil removal of textiles,
(viii) preventing resoiling of textiles, and/or
(ix) reducing the shrinkage of textiles.
   4. The use of any one of items 1 to 3, wherein treating or finishing textiles includes improving or maintaining the properties of textiles or introducing properties to textiles.
   5. The use of item 4, wherein improving or maintaining the properties of textiles or introducing properties to textiles encompasses
(i) preventing the loss of tensile strength of textiles,
(ii) preventing the loss of elasticity of textiles,
(iii) improving the shape retention of textiles,
(iv) reducing the shrinkage of textiles,
(v) improving the mechanical resistance of textiles,
(vi) reducing wrinkling/increasing the wrinkling resistance of textiles,
(vii) facilitating ironing of textiles,
(viii) improving the haptics, preferably smoothness, of textiles,
(ix) improving the water vapour permeability of textiles,
(x) improving the wettability of textiles,
(xi) introducing UV ray blocking to textiles,
(xii) improving the breathability of textiles, and/or
(xiii) improving the abrasion resistance of textiles.
   6. The use of any one of items 1 to 5, wherein treating or finishing textiles includes restoring textiles.
   7. The use of item 6, wherein restoring textiles encompasses
(i) smoothing of textiles,
(ii) dissolving fluffs and nodules in textiles,
(iii) reducing pilling of textiles,
(iv) reducing fluffing of textiles, and/or
(v) filling cavities within the fibers of textiles.
   8. The use of any one of items 1 to 7, wherein treating or finishing textiles includes improving the microbiological properties of textiles.
   9. The use of item 8, wherein improving the microbiological properties of textiles encompasses
(i) preventing bad odour of textiles,
(ii) blocking bad odour of textiles, and/or
(iii) preventing or inhibiting microbial growth in textiles.
   10. The use of any one of items 1 to 9, wherein treating or finishing textiles includes improving the olfactory properties of textiles.
   11. The use of item 10, wherein improving the olfactory properties of textiles encompasses
(i) improving the perfume impression of textiles, and/or
(ii) controlling release of perfumes from textiles.
   12. The use of any one of items 1 to 11, wherein the structural polypeptide is comprised in an aqueous formulation.
   13. The use of item 12, wherein the concentration of the structural polypeptide in the aqueous formulation is in the range of 0.01% by weight to 20% by weight, preferably 0.1% by weight to 15% by weight, and more preferably 10% by weight to 10% by weight.
   14. The use of items 12 or 13, wherein the aqueous formulation has a gel-like structure.
   15. The use of item 14, wherein the gel-like structure is a hydrogel.
   16. The use of any one of items 1 to 15, wherein the structural polypeptide is a silk polypeptide.
   17. The use of item 16, wherein the silk polypeptide is a recombinant silk polypeptide.
   18. The use of items 16 or 17, wherein the silk polypeptide comprises or consists of (at least two identical) repetitive units.
   19. The use of item 18, wherein the repetitive units are independently selected from the group consisting of module C having an amino acid sequence according to SEQ ID NO: 1 or variants thereof, module C^{Cys} having an amino acid sequence according to SEQ ID NO: 2 or variants thereof, module C^{K} having an amino acid sequence according to SEQ ID NO: 3 or variants thereof, and module C^{Lys} having an amino acid sequence according to SEQ ID NO: 4 or variants thereof.
   20. The use of any one of items 16 to 19, wherein the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96.
   21. The use of item 20, wherein the silk polypeptide is selected from the group consisting of C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys} (C)₃₂C^{Cys}, (C)₄₈C^{Cys}, (C)₂C^{K}, (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys}, (C)₆C^{Lys}, (C)₈C^{Lys}, (C)₁₆C^{Lys}, (C)₃₂C^{Lys}, (C)₄₈C^{Lys} C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}₂, C^{Cys}₄, C^{Cys}₆, C^{Cys}₈, C^{Cys}₁₆, C^{Cys}₃₂, C^{Cys}₄₈, C^{K}₂, C^{K}₄, C^{K}₆, C^{K}₈, c^{K}₁₆, C^{K}₃₂, C^{K}₄₈, C^{Lys}₂, C^{Lys}₄, C^{Lys}₆, C^{Lys}₈, C^{Lys}₁₆, C^{Lys}₃₂, and C^{Lys}₄₈.
   22. The use of any one of items 1 to 21, wherein the textiles are selected from the group consisting of woven, non-woven or knitted textiles.
   23. The use of any one of items 1 to 22, wherein the textiles are animal derived textiles, plant derived textiles, synthetic textiles, or blends thereof.
   24. The use of item 23, wherein the animal derived textiles are selected from the group consisting of wool and silk textiles, the plant derived textiles are selected from the group consisting of cotton, flax, rayon, and seaweed textiles, or the synthetic textiles are selected from the group consisting of nylon, polyester, and spandex textiles.
   25. A textile treated or finished with a structural polypeptide.
   26. The textile of item 25, wherein the structural polypeptide is a silk polypeptide.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURE

The following figures are merely illustrative of the present invention and should not be construed to limit the scope of the invention as indicated by the appended claims in any way.

FIGS. 1A-1B: Pictures of the apparatus for finishing of fibers during finishing of cotton fiber.

Left: Uptake-Reel (UR), Feed-Reel (FR), Immersion-Tank 1 (IT1), Coating-Roll 1 (CR1), Switching-Shaft 1(SS1) and Switching-Roll 1. Right: Switching-Roll 2 (SR2), Immersion Tank 2 (IT2), Coating-Roll 2 (CR2), Switching-Shaft 2 (SS2) and Switching-Roll 3 (SR3).

FIG. 2: Weaving frame for production of textile fabrics.

FIGS. 3A-3B: LSM (laser scanning microscope) pictures of non-treated (left) and treated wool fiber (right). The treatment of the wool fiber with silk leads to the covering of cuticles. The smoother surface changes the reflection of light from diffuse to more specular. Consequently, the treated textiles show an improvement in gloss impression and appear more shining.

FIGS. 4A-4B: LSM pictures of non-treated (left) and treated nylon fibers (right). The treatment of the nylon fiber with silk leads to a smoother surface. The smoother surface changes the reflection of light from diffuse to more specular. Consequently, the treated textiles show an improvement in gloss impression and appear more shining.

FIGS. 5A-5B: LSM pictures of non-treated (left) and treated wool fiber (right). The treatment of the fiber with silk leads to the covering of cuticles. The smoother surface leads to increased softness and reduced scratchiness.

FIGS. 6A-6B: LSM pictures of non-treated (left) and treated nylon fibers (right). The treatment of the fiber with silk leads to a smoother surface increasing the smoothness of the textiles and leading to softer touch.

FIGS. 7A-7B: LSM pictures of non-treated (left) and treated nylon fibers (right). The treatment of the fiber with silk leads to a smoother surface increasing the smoothness of the textiles and leading to softer touch. In addition, the silk coverage is able to reduce pilling and fluffing of textiles.

FIGS. 8A-8B: LSM pictures of non-treated (left) and treated wool fiber (right). The treatment of the fiber with silk leads to the covering of cuticles and consequently to increased smoothness, softer touch and less scratchy textiles. In addition, silk is able to reduce pilling and fluffing of textiles.

FIGS. 9A-9B: Adhesion of silk capsules with perfume oil to cotton thread (left). Silk capsules on cotton thread after drying (right).

FIGS. 10A-10B: Results of in vitro-tests. Samples were applied onto sterile culture plates and incubated for 3 days. High microbial load could be observed on plates with polyester samples (A), whereas no microbial growth could be observed on plates with silk polypeptide (C16) samples (B).

### EXAMPLES

The examples given below are for illustrative purposes only and do not limit the invention described above in any way.

### Example 1: Preparation of C₁₆ Silk Hydrogels

### a) Preparation of C₁₆ Protein:

The C₁₆ protein (SEQ ID NO: 6) was prepared as described in WO 2006/008163.

### b) Preparation of Protein Solutions:

For the preparation of protein solutions, the protein was dissolved in 6 M GdmSCN and 50 mM Tris/HCl, pH 8.0. In order to remove the GdmSCN, protein solutions were dialyzed against 10 mM Tris/HCl, pH 9.0 using a Spectra/Por Dialysis Membrane with a MWCO of 6000-8000. The solution was centrifuged at 2,860×g for 10 min at room temperature.

At a volume of the protein solution of >500 mL, the GdmSCN was removed and the protein solution was concentrated without dialysis using a crossflow unit (Sartorius AG, Gottingen) with SARTOCON Slice Cassettes (Filter material: Hydrosat with 10 kDa cut off).

The C₁₆ protein concentration was determined by measuring the absorbance at 276 nm using the UV/Vis spectroscopy (Beckman Coulter). The final protein concentration in protein solution was between 0.5 and 9% (w/w) according to the respective application.

### c) Preparation of Protein Hydrogels:

For the preparation of protein solutions, the protein was dissolved in 6 M GdmSCN and 50 mM Tris/HCl, pH 8.0. In order to remove the GdmSCN, protein solutions were dialyzed against 10 mM Tris/HCl, pH 9.0 using a Spectra/Por Dialysis Membrane with a MWCO of 6000-8000. The solution was centrifuged at 2,860×g for 10 min at room temperature.

At a volume of the protein solution of >500 mL, the GdmSCN was removed and the protein solution was concentrated without dialysis using a crossflow unit (Sartorius AG, Gottingen) with SARTOCON Slice Cassettes (Filter material: Hydrosat with 10 kDa cut off).

The C₁₆ protein concentration was determined by measuring the absorbance at 276 nm using the UV/Vis spectroscopy (Beckman Coulter). The final protein concentration in protein solution was between 0.5 and 9% (w/w) according to the respective application. Either the protein solutions were autoclaved at 121° C. for 15 min or SymDiol® (Symrise, Holzminden, Germany) was added to a final concentration of 1% to avoid microbial growth during subsequent storage. The samples were stored for 1 hour to 36 months at temperatures of 4° C. to 30° C. for maturation to form hydrogel structures.

### Example 2: Treatment and Finishing of Fibers and Textiles

### a) Treatment and Finishing of Fibers Via Bathing:

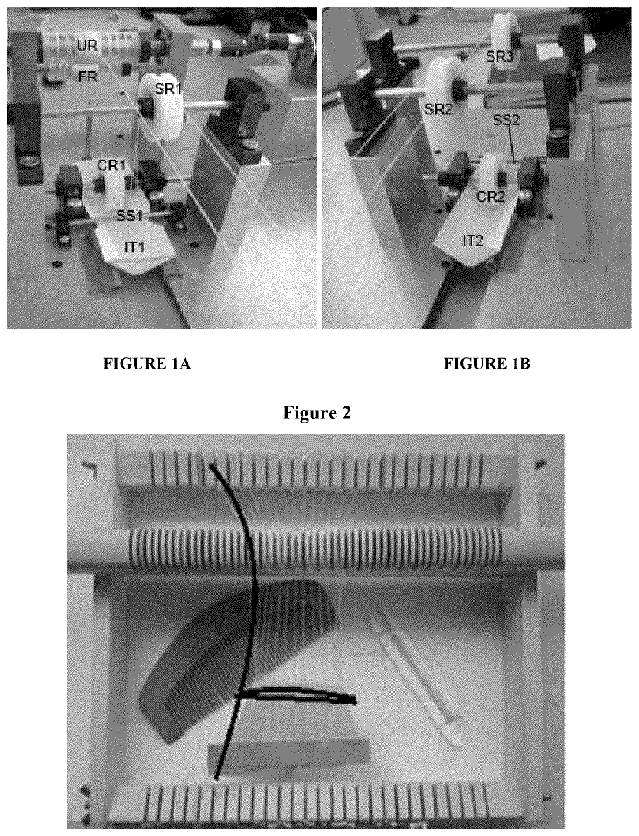

The treatment and finishing of fibers (wool, cotton, nylon, polyester) were performed with a constructed apparatus (see FIG. 1). The fibers were dragged twice through two immersion tanks filled with 15 mL of protein solution respectively (protein concentration 1 and 2% w/w). Drying was either performed at room temperature (20-30° C.) or in a convection oven with a first drying step at 60-110° C. for 30-1200 s at 400-1200 rpm and a second drying step at 130-180° C. for 30-300 s at 400-1200 rpm. After drying, the fibers were dragged again twice through washing water or a post treatment solution (500 mM potassium phosphate pH 8.0) and subsequent washing step with washing water. The dragging speed was set to 2-12 m/min. Drying was either performed at room temperature (20-30° C.) or in a convection oven with a first drying step at 60-110° C. for 30-1200 s at 400-1200 rpm and a second drying step at 130-180° C. for 30-300 s at 400-1200 rpm.

### b) Treatment and Finishing of Textiles Via Dipping:

The fibers and textiles were immersed into protein solution in a beaker with 50 mL protein solution (protein concentration 2% w/w), removed and subsequently dried at room temperature (20-30° C.).

### c) Treatment and Finishing of Textiles Via Spraying:

The protein solution (protein concentration 1-2% w/w) was transferred to a spray can or spraying device (Bottle ELLIPS 50 ml Nr. 00041, Hartwig Kröger GmbH with a spraying device Zerstäuberpumpe 00355+00352, Hartwig Kröger GmbH). The protein solution was sprayed on textiles till full saturation. The fabrics were dried at room temperature (20-30° C.).

d) Preparation of Textile Fabrics with Treated and Finished Fibers:

The textile fabrics were woven with a modified weaving frame (see FIG. 2). The size of the textile fabrics was defined to 45 cm².

### Example 3: Improving or Maintaining the Optics of Different Textiles

### a) Improving or Maintaining the Optics of Cotton Fibers (Improvement of Gloss Impression and Shining of Textiles):

Cotton fibers finished with C₁₆ (SEQ ID NO: 6) according to example 2 a) were used. The final protein amount on the fiber was determined to 1.2% (w/w). Textiles with a surface of 45 cm² were woven according to example 2 d)—one with non-treated fiber and one with treated fiber. Due to the very good wettability of cotton—because of the structured surface of cotton monofilaments and the arrangement of cotton monofilaments in fibers—the silk protein solution could be applied very well to cotton textiles. The textiles were tested by a pool of 15 people. The criteria of the test were general optics and the overall quality of the textiles and in particular the gloss impression and shining of textiles. 10 of the people voted for the treated textile to have better optics (improvement of gloss impression and shining of textiles). 0 of the people voted for the non-treated textile to have better optics. 5 of the people abstained from voting.

### b) Improving or Maintaining the Optics of Wool Fibers (Improvement of Gloss Impression and Shining of Textiles):

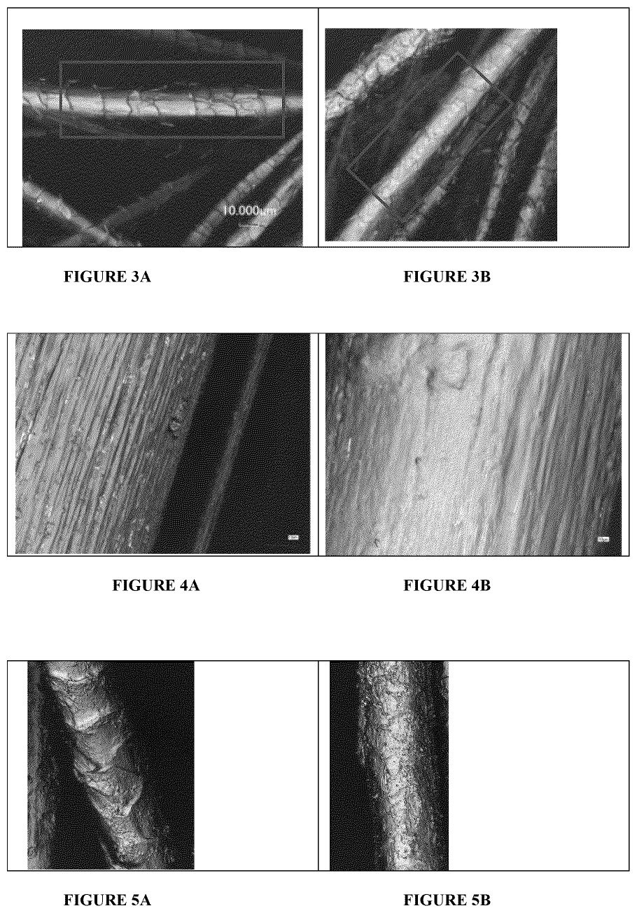

Wool fibers finished with C₁₆ (SEQ ID NO: 6) according to example 2 a) were used. The final protein amount on the fiber was determined to 10.5% (w/w). Textiles with a surface of 45 cm2 were woven according to example 2 d)—one with non-treated fiber and one with treated fiber. Wool showed a very good wettability, because of the structured surface of wool monofilaments and the arrangement of monofilaments in the fiber. The textiles were tested by a pool of 15 people. The criteria of the test were general optics and the overall quality of the textiles and in particular the gloss impression and shining of textiles. 6 of the people voted for the treated textile to have better optics. 2 of the people voted for the non-treated textile to have better optics. 8 of the people abstained from voting. In particular the cuticles of the fiber were covered by silk leading to an improvement in gloss impression and a more shining textile (see FIG. 3).

### c) Improving or Maintaining the Optics of Nylon Fibers (Improvement of Gloss Impression and Shining of Textiles):

Colored nylon fibers finished with C₁₆ (SEQ ID NO: 6) according to example 2 a) were used. The final protein amount on the fiber was determined to 5.0% (w/w). The colored nylon single yarn showed a very good wettability, because of the structured surface of the monofilaments. Textiles with a surface of 45 cm² were woven according to example 2 d)—one with non-treated fiber and one with treated fiber. The textiles were tested by a pool of 15 people. The criteria of the test were general optics and the overall quality of the textiles and in particular the gloss impression and shining of textiles. 8 of the people voted for the treated textile to have better optics. 0 of the people voted for the non-treated textile to have better optics. 7 of the people abstained from voting. The reason for the improved optics is most likely the smoothened surfaced of the nylon fiber after treatment (see FIG. 4). In particular the treated textiles were rated to have an improved gloss impression and to be more shining.

### d) Improving or Maintaining the Optics of Polyester Fibers (Improvement of Gloss Impression and Shining of Textiles):

Colored polyester fibers finished with C₁₆ (SEQ ID NO: 6) according to example 2 a) were used. The colored polyester single yarn showed a good wettability. The final protein amount on the fiber was determined to 3.4% (w/w). Textiles with a surface of 45 cm² were woven according to example 2 d)—one with non-treated fiber and one with treated fiber. The textiles were tested by a pool of 15 people. The criteria of the test were general optics and the overall quality of the textiles and in particular the gloss impression and shining of textiles. 9 of the people voted for the treated textile to have better optics. 5 of the people voted for the non-treated textile to have better optics. 1 of the people abstained from voting. In particular the treated textiles were rated to have an improved gloss impression and to be more shining.

### Example 4: Improving or Maintaining the Properties of Textiles or Introducing Properties to Different Textiles

### a) Improving or Maintaining the Properties or Introducing Properties to Cotton (Improvement of Haptics, Elasticity, Wrinkling, Smoothness, Soft Touch and Scratchiness):

Cotton fibers finished with C₁₆ (SEQ ID NO: 6) according to example 2 a) were used. The final protein amount on the fiber was determined to 1.2% (w/w). Textiles with a surface of 45 cm² were woven according to example 2 d)—one with non-treated fiber and one with treated fiber. The textiles were tested by a pool of 15 people. The criteria of the test were general haptics and the overall quality of the textiles and in particular the elasticity, wrinkling behavior, smoothness, soft touch and scratchiness. 11 of the people voted for the treated textile to have better haptics. 4 of the people voted for the non-treated textile to have better haptics. 0 of the people abstained from voting. In particular, the textiles were rated to show increased elasticity and had less tendency to wrinkle. The predominant portion of people mentioned the soft touch and smoothness of the textile leading to less scratchy feeling.

### b) Improving or Maintaining the Properties or Introducing Properties to Wool (Improvement of Haptics, Elasticity, Wrinkling, Smoothness, Soft Touch and Scratchiness, Abrasion Resistance):

Wool fibers finished with C₁₆ (SEQ ID NO: 6) according to example 2 a) were used. The final protein amount on the fiber was determined to 10.5% (w/w). Textiles with a surface of 45 cm² were woven according to example 2 d)—one with non-treated fiber and one with treated fiber. The textiles were tested by a pool of 15 people. The criteria of the test were general haptics and the overall quality of the textiles and in particular the elasticity, wrinkling behavior, smoothness, soft touch and scratchiness. 9 of the people voted for the treated textile to have better haptics. 5 of the people voted for the non-treated textile to have better haptics. 1 of the people abstained from voting. In particular, the softness of the treated textile was rated higher and the textile was rated to be less scratchy, which is most likely caused by covering the cuticles with silk (see FIG. 5). In addition, the treated textile showed a lower tendency to wrinkle and a higher elasticity.

### c) Improving or Maintaining the Properties or Introducing Properties to Wool (Abrasion Resistance):

To verify the influence of silk treatment and finishing on the abrasion resistance, a Martindale abrasion test was performed according to DIN53863 with the wool fabrics. The reference—non-treated wool fabric—reached 490 cycles whereas the silk treated fabric reached 860 cycles. Hence, the abrasion resistance could be increased by treatment and finishing of wool fabrics with silk solution.

### d) Improving or Maintaining the Properties or Introducing Properties to Nylon (Improvement of Haptics, Elasticity, Wrinkling, Smoothness, Soft Touch and Scratchiness):

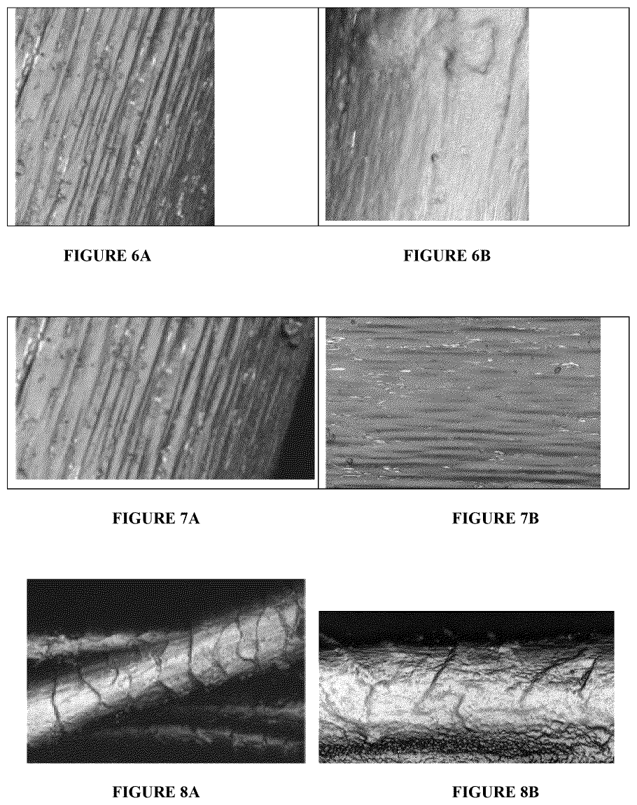

Nylon fibers finished with C₁₆ (SEQ ID NO: 6) according to example 2 a) were used. The final protein amount on the fiber was determined to 5.0% (w/w). Textiles with a surface of 45 cm² were woven according to example 2 d)—one with non-treated fiber and one with treated fiber. The textiles were tested by a pool of 15 people. The criteria of the test were general haptics and the overall quality of the textiles and in particular the elasticity, wrinkling behavior, smoothness, soft touch and scratchiness. 11 of the people voted for the treated textile to have better haptics. 0 of the people voted for the non-treated textile to have better haptics. 4 of the people abstained from voting. In particular the softness of the treated textiles was rated, which is most likely caused by smoothening the surface of nylon fibers by the treatment (see FIG. 6). In addition, the treated textile showed a lower tendency to wrinkle.

### e) Improving or Maintaining the Properties or Introducing Properties to a Nylon Fabric (Abrasion Resistance):

To verify the influence of silk treatment and finishing on the abrasion resistance, a Martindale abrasion test was performed according to DIN53863 with the nylon fabrics. The reference—non-treated nylon fabric—reached 2920 cycles whereas the silk treated fabric reached 3910 cycles. Hence, the abrasion resistance could be increased by treatment and finishing of wool fabrics with silk solution.

f) Improving or Maintaining the Properties or Introducing Properties to Polyester (Improvement of Haptics. Elasticity. Wrinkling. Smoothness, Soft Touch and Scratchiness):

Polyester fibers finished with C₁₆ (SEQ ID NO: 6) according to example 2 a) were used. The final protein amount on the fiber was determined to 3.4% (w/w). Textiles with a surface of 45 cm² were woven according to example 2 d)—one with non-treated fiber and one with treated fiber. The textiles were tested by a pool of 15 people. The criteria of the test were general haptics and the overall quality of the textiles and in particular the elasticity, wrinkling behavior, smoothness, soft touch and scratchiness. 11 of the people voted for the treated textile to have better haptics. 3 of the people voted for the non-treated textile to have better haptics. 1 of the people abstained from voting. In particular the softness of the treated textiles was rated, which is most likely caused by smoothening the surface of polyester fibers by the treatment. In addition, the treated textile showed a lower tendency to wrinkle.

### Example 5: Restoring Textiles

Textiles suffer from reduced performance with regard to rough surface of textiles, building of fluffs and nodules and pilling of textiles after usage. The suitability of treatment of textiles with silk for restoring textiles was shown, as treatment with silk leads to smoothing the surface of textiles, reducing pilling and fluffing, nodule formation of textiles.

### a) Restoring the Synthetic Polymer-Based Fibers:

Nylon fibers finished with C₁₆ (SEQ ID NO: 6) according to example 2 a) were used. The fibers were analyzed via microscopy before and after treatment.

It could be shown that silk is able to smoothen the surface of synthetic polymer-based fibers like nylon fibers leading to increased smoothness, softer touch and less scratchy textiles. In addition, the covering with silk reduces pilling and fluffing of the textiles (see FIG. 7).

### b) Restoring Natural Fibers (Surface Restoration):

Wool fibers finished with C₁₆ (SEQ ID NO: 6) according to example 2 a) were used. The fibers were analyzed via microscopy before and after treatment.

Silk is able to cover the cuticles of natural fibers like wool leading to leading to increased smoothness, softer touch and less scratchy textiles. In addition, the covering with silk reduces pilling and fluffing of the textiles (see FIG. 8).

### c) Restoring Natural Fibers (Tensile Strength):

A treating solution was prepared by diluting 5 mL of C₁₆ protein (single C module: SEQ ID NO: 1, C₁₆ protein: SEQ ID NO: 6) aqueous solution (1% protein concentration) with 495 mL of deionized water in a beaker, leading to a final protein concentration of 0.01% in the treating solution. Deionized water was used as treating solution for reference samples. Wool fibers were dipped into the beakers with treating solution or water for 1-30 minutes under stirring with a magnetic stirrer. The wool fibers were then washed in 10 mL of deionized water in a beaker under constant stirring with a magnetic stirrer, leading to estimated final protein concentrations of 0.0001% to 0.001% of C₁₆ protein in the washing solution. Subsequently, the wool fibers were dried at 20-30° C. for 1-8 h and used for testing of tensile strength.

The fibers were analyzed via tensile testing (Zwick BT1-FK0.5N.D14 from Zwick/Roell GmbH & Co. KG) to determine the breaking force according to DIN EN ISO 2062 at a pulling speed of 250 mm/min and an effective clamping length of 80 mm. The tensile strength was standardized to the diameter of the measured fiber (analyzed by LSM) before and after treatment. The mean tensile strength was calculated from 10 measurements each.

Some of the samples were additionally treated by ironing at 130-150° C. for 10 sec. The samples were analyzed after 20 cycles of ironing, treatment with water (sample 1) or treatment with C₁₆ (SEQ ID NO: 6) according to example 2 a) (sample 2) and drying at 20-30° C. for 1-16 h.

The following samples were prepared and tested to analyze the restorative effect of silk on wool fibers:

| | Standardized |
|---|---|
| Sample | breaking force [%] |
| Reference: Non-treated wool | 100 ± 22% |
| fiber before ironing | |
| Sample 1: Water-treated wool after | 58 ± 14% |
| 20 cycles of ironing, washing and drying | |
| Sample 2: Wool after ironing and treated | 86 ± 12% |
| with C16 after ironing | |

In conclusion, the restorative effect of silk with regard to retaining tensile strength was shown, as the treatment with C16 solution after each ironing cycle lead to an increase in standardized breaking force of wool fibers after 20 ironing cycles of 28% compared to samples which were only treated with water.

### Example 6: Improving the Microbiological Properties of Textiles

### a) Improving Microbial Properties of Textiles Using Silk Fibers:

Textile samples of C₄₈C^{Cys} silk protein fibers were cut into 15×15 mm pieces (20 pieces). Commercially available polyester textile was used as control and cut into 15×15 mm pieces. The analysis was carried out according to a modified procedure of ISO 22196. Within this setup, a thin film of bacteria (Staphylococcus epidermidis DSM 18857) solution (1.25×104/cm²) was given directly onto each sample. A foil (Stomacher-Bags) was applied to each well, to prevent dehydration. For blank sample, bacteria were removed using ultrasonic and vortex technique, immediately after inoculation. Bacteria were counted (colony forming units—CFU, t0-value). Another set was incubated in wet conditions for 24 hours at 37° C. Bacteria estimation was carried out like described before (t24-value).

The silk protein fiber textile showed a reduction of bacterial adhesion of >99.99% which equals a log reduction of >4 in comparison to non-treated polyester textile. The reduction of bacterial adhesion is shown in FIG. 10.

Hence, the suitability of silk material to improve microbial properties of textiles, i.e. reducing adhesion of film forming microorganisms was shown.

### b) Improving Microbial Properties of Textiles by Treating and Finishing of Materials:

Silicon foil was used as model material for synthetic polymer-based fibers. The material was cut into 15×15 mm pieces. The samples were stored into a Falcon tube. Samples were autoclaved in 50 mL Falcon tubes covered with aluminum foil for 20 min at 121° C. Fabric pieces were placed in sterile petri plates and allowed to dry under sterile bench for 1-2 h. Half of the autoclaved samples were dipped into a falcon filled with 1% sterilized (20 min at 121° C.) C16 silk hydrogel for 5 minutes—according to example 2 b). After dipping, samples were dried at room temperature under sterile conditions over night. The other half of the autoclaved samples served as control. These were dipped into a falcon filled with sterilized deionized water (20 min 121° C.) for 5 minutes. After dipping, samples were dried at room temperature under sterile conditions over night.

The analysis was carried out according to a modified procedure of ISO 22196. Within this setup, a thin film of bacteria (Staphylococcus epidermidis DSM 18857) solution (1.25×104/cm²) was given directly onto each sample. A foil (Stomacher-Bags) was applied to each well, to prevent dehydration. For blank sample, bacteria were removed using ultrasonic and vortex technique, immediately after inoculation. Bacteria were counted (colony forming units—CFU, t0-value).

Another set was incubated in wet conditions for 24 hours at 37° C. Bacteria estimation was carried out like described before (t24-value).

The treatment and finishing of the material with protein lead to a reduction of bacterial adhesion of 93.16% on silicone in comparison to the non-treated sample which equals a log reduction of 1.16.

These finding show the ability of silk treatment for reduction of bacterial adhesion on textiles and preventing or inhibiting microbial growth in textiles. Consequently, the treatment with silk was shown to be able to potentially prevent bad odor and blocking bad odor of textiles caused by microbial growth in textiles.

### Example 7: Improving the Olfactory Properties of Textiles

A concept for treating textiles with a silk solution containing fragrances was developed.

a) Preparation of Silk Solution from C₁₆ (SEQ ID NO: 6) with Perfume Oil:

Silk hydrogel with 3% C₁₆ silk protein was produced according to example 2c). The hydrogel was treated with an ultra turrax (12,000 rpm, 1 min) until the hydrogel got flowable. Subsequently, 1 mL of perfume oil was added to 49 mL of the flowable hydrogel. The mixture was again treated with an ultra turrax (12,000 rpm, 1 min). The result was a flowable silk hydrogel with perfume oil.

b) Preparation of Silk Solution from C₁₆^{K} with Perfume Oil:

Silk hydrogel with 3% C₁₆^{K} silk protein was produced according to example 2c). The hydrogel was treated with an ultra turrax (12,000 rpm, 1 min) until the hydrogel got flowable. Subsequently, 1 mL of perfume oil was added to 49 mL of the flowable hydrogel. The mixture was again treated with an ultra turrax (12,000 rpm, 1 min). The result was a flowable silk hydrogel with perfume oil.

c) Treatment of Cotton Thread with Silk Solution from C₁₆^{K} with Perfume Oil:

0.5 g of silk solution was suspended in 44.5 g of deionized water and 5 g of cationic polymers (Polyquatemium-7 (Lubrizol Advanced Materials Inc., 9911 Brecksville Road, Cleveland, Ohio)). The cationic polymers were selected from the group of polyquaterniums, which are organic chemicals. These polymers have quaternary ammonium centers in the polymer. They are positively charged and neutralize negative charges and are commonly used in hair or personal care. Their positive charge can ionically link them to materials, like hair or skin.

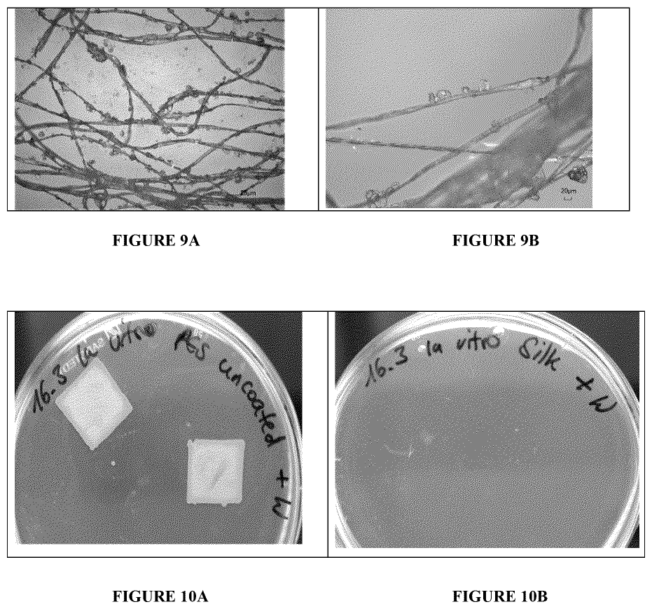

The mixture was shaken. 20 μL of the suspension was put on a glass slide. A single cotton thread was dipped in the drop of the described suspension of silk solution, water and polymers. As reference 20 μL of a suspension containing only perfume oil, deionized water and cationic polymers was used. The threads were dipped into a drop of 20 μL of the reference solution. An adhesion of the silk on the cotton thread could be observed (see FIG. 9A) The silk films were stable after drying at room temperature. The perfume oil was fixed in the silk film, which forms a diffusion barrier leading to slower release of the fragrances. These findings show that treatment with silk is suitable to fix perfumes with fragrances to textiles leading to an improved perfume impression of textiles and a controlled long-term release of perfumes from textiles.

In particular, the perfume impression of samples with silk directly after treatment was rated to be better compared to the reference, as the initial smell was reduced and rated to be more compelling. In addition, the perfume smell was shown to be longer lasting because of the controlled release effect compared to the reference.

d) Treatment of Wool, Polyester and Nylon Threads with Silk Solution from C₁₆ with Perfume Oil:

0.5 g of silk solution was suspended in 44.5 g of deionized water and 5 g of cationic polymers (Polyquatemium-7 (Lubrizol Advanced Materials Inc., 9911 Brecksville Road, Cleveland, Ohio)). The cationic polymers were selected from the group of polyquaterniums, which are organic chemicals. These polymers have quaternary ammonium centers in the polymer. They are positively charged and neutralize negative charges and are commonly used in hair or personal care. Their positive charge can ionically link them to materials, like hair or skin.

The mixture was shaken. 20 μL of the suspension was put on a glass slide. Single wool, polyester and nylon threads were dipped in the drop of the described suspension of silk solution, water and polymers. As reference 20 μL of a suspension containing only perfume oil, deionized water and cationic polymers was used. The threads were dipped into a drop of 20 μL of the reference solution. An adhesion of the silk on the threads could be observed (see FIG. 9A, B). The silk films were stable after drying at room temperature. The perfume oil was fixed in the silk film, which forms a diffusion barrier leading to slower release of the fragrances. These findings show that treatment with silk is suitable to fix perfumes with fragrances to textiles leading to an improved perfume impression of textiles and a controlled long-term release of perfumes from textiles.

In particular, the perfume impression of samples with silk directly after treatment was rated to be better compared to the reference, as the initial smell was reduced and rated to be more compelling. In addition, the perfume smell was shown to be longer lasting because of the controlled release effect compared to the reference.

1. A method of using a silk polypeptide for treating or finishing textiles.

2. The method of claim 1,
wherein treating or finishing textiles includes improving or maintaining the optics of textiles, and
wherein improving or maintaining the optics of textiles encompasses
(i) reducing the graying of textiles,
(ii) improving the whiteness of textiles,
(iii) reducing the discoloration or fading of textiles,
(iv) preventing the change in colour impression of textiles,
(v) improving the gloss impression or shining of textiles,
(vi) improving the luminosity of textiles,
(vii) improving the soil removal of textiles,
(viii) preventing resoiling of textiles, and/or
(ix) reducing the shrinkage of textiles.

3. The method of claim 1,
wherein treating or finishing textiles includes improving or maintaining the properties of textiles or introducing properties to textiles, and
wherein improving or maintaining the properties of textiles or introducing properties to textiles encompasses
(i) preventing the loss of tensile strength of textiles,
(ii) preventing the loss of elasticity of textiles,
(iii) improving the shape retention of textiles,
(iv) reducing the shrinkage of textiles,
(v) improving the mechanical resistance of textiles,
(vi) reducing wrinkling/increasing the wrinkling resistance of textiles,
(vii) facilitating ironing of textiles,
(viii) improving the haptics, preferably smoothness, of textiles,
(ix) improving the water vapour permeability of textiles,
(x) improving the wettability of textiles,
(xi) introducing UV ray blocking to textiles,
(xii) improving the breathability of textiles, and/or
(xiii) improving the abrasion resistance of textiles.

4. The method of claim 1,
wherein treating or finishing textiles includes restoring textiles, and
wherein restoring textiles encompasses
(i) smoothing of textiles,
(ii) dissolving fluffs and nodules in textiles,
(iii) reducing pilling of textiles,
(iv) reducing fluffing of textiles, and/or
(v) filling cavities within the fibers of textiles.

5. The method of claim 1,
wherein treating or finishing textiles includes improving the microbiological properties of textiles, and
wherein improving the microbiological properties of textiles encompasses
(i) preventing bad odour of textiles,
(ii) blocking bad odour of textiles, and/or
(iii) preventing or inhibiting microbial growth in textiles.

6. The method of claim 1,
wherein treating or finishing textiles includes improving the olfactory properties of textiles, and
wherein improving the olfactory properties of textiles encompasses
(i) improving the perfume impression of textiles, and/or
(ii) controlling release of perfumes from textiles.

7. The method of claim 1, wherein the silk polypeptide is comprised in an aqueous formulation.

8. The method of claim 7, wherein the concentration of the silk polypeptide in the aqueous formulation is in the range of 0.01% by weight to 20% by weight.

9. The method of claim 7, wherein the aqueous formulation has a gel-like structure.

10. The method of claim 9, wherein the gel-like structure is a hydrogel.

11. The method of claim 1,
wherein the silk polypeptide comprises or consists of (at least two identical) repetitive units, and
wherein the repetitive units are independently selected from the group consisting of module C having an amino acid sequence according to SEQ ID NO: 1 or variants thereof,
module C^{Cys} having an amino acid sequence according to SEQ ID NO: 2 or variants thereof,
module C^{K} having an amino acid sequence according to SEQ ID NO: 3 or variants thereof, and module C^{Lys} having an amino acid sequence according to SEQ ID NO: 4 or variants thereof.

12. The method of claim 11,
wherein the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96, and
wherein the silk polypeptide is selected from the group consisting of C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys}, (C)₃₂C^{Cys}, (C)₄₈C^{Cys} (C)₂C^{K}, (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys}, (C)₆C^{Lys} (C)₈C^{Lys}, (C)₁₆C^{Lys} (C)₃₂C^{Lys} (C)₄₈C^{Lys} C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}₂, C^{Cys}₄, C^{Cys}₆, C^{Cys}₈, C^{Cys}₁₆, C^{Cys}₃₂, C^{Cys}₄₈, C^{K}₂, C^{K}₄, C^{K}₆, C^{K}₈, C^{K}₁₆, C^{K}₃₂, C^{K}₄₈, C^{Lys}₂, C^{Lys}₄, C^{Lys}₆, C^{Lys}₈, C^{Lys}₁₆, C^{Lys}₃₂, and C^{Lys}₄₈.

13. The method of claim 1,
wherein the textiles are selected from the group consisting of woven, non-woven or knitted textiles, and
wherein the textiles are animal derived textiles, plant derived textiles, synthetic textiles, or blends thereof.

14. A textile treated or finished with a silk polypeptide.

15. The textile of claim 14, wherein the textile is treated or finished with a silk polypeptide hydrogel.

16. The method of claim 1, wherein said method comprises the steps of:
(i) diluting a dose of an aqueous formulation comprising a silk polypeptide with an aqueous solution to obtain a treating solution, and
(ii) applying the treating solution so formed to the textiles.

17. The method of claim 16, wherein the treating solution is applied by
bathing the textiles in the treating solution,
spraying the treating solution onto the textiles,
nebulizing the treating solution onto the textiles, and/or
dipping the textiles in the treating solution.

18. The method of claim 8, wherein the concentration of the silk polypeptide in the aqueous formulation is in the range of 0.1% by weight to 15% by weight.

19. The method of claim 18, wherein the concentration of the silk polypeptide in the aqueous formulation is in the range of 1% by weight to 10% by weight.

20. The method of claim 13, wherein
the animal derived textiles are selected from the group consisting of wool and silk textiles,
the plant derived textiles are selected from the group consisting of cotton, flax, rayon, and seaweed textiles, or
the synthetic textiles are selected from the group consisting of nylon, polyester, and spandex textiles.


SEQUENCE LISTING (8 sequences)

SEQ ID NO: 1 - protein, 35 aa, synthetic construct
  REGION 1..35: note = synthetic
  DOMAIN 1..35: note = Module C (ADF-4)
GSSAAAAAAAASGPGGYGPENQGPSGPGGYGPGGP

SEQ ID NO: 2 - protein, 35 aa, synthetic construct
  REGION 1..35: note = synthetic
  DOMAIN 1..35: note = Module Ccys
GSSAAAAAAAASGPGGYGPENQGPCGPGGYGPGGP

SEQ ID NO: 3 - protein, 35 aa, synthetic construct
  REGION 1..35: note = synthetic
  REGION 1..35: note = domain - Module Ck
GSSAAAAAAAASGPGGYGPKNQGPSGPGGYGPGGP

SEQ ID NO: 4 - protein, 35 aa, synthetic construct; identical to SEQ ID NO: 3
  REGION 1..35: note = synthetic
  REGION 1..35: note = Domain - Module Clys

SEQ ID NO: 5 - protein, 280 aa, synthetic construct
  REGION 1..280: note = synthetic
  REPEAT 1..280: note = C8
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 6 - protein, 560 aa, synthetic construct
  REGION 1..560: note = synthetic
  REPEAT 1..560: note = C16
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 7 - protein, 1120 aa, synthetic construct
  REGION 1..1120: note = synthetic
  REPEAT 1..1120: note = C32
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 8 - protein, 1680 aa, synthetic construct
  REGION 1..1680: note = synthetic
  REPEAT 1..1680: note = C48
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP